(19) United States
(12) Patent Application Publication
(10) Pub. No.: US 2026/0137800 A1
(43) Pub. Date: May 21, 2026

(54) IMMUNOCAPTURE METHODS TO ENRICH FOR ENGINEERED EXTRACELLULAR VESICLES

(71) Applicant: The General Hospital Corporation, Boston, MA (US)

(72) Inventors: Koen Breyne, Reading, MA (US); Xandra O. Breakefield, Newton Center, MA (US)

(21) Appl. No.: 19/121,046

(22) PCT Filed: Oct. 13, 2023
(86) PCT No.: PCT/US2023/076828

§ 371 Date: Apr. 14, 2025

Related U.S. Application Data
(60) Provisional application No. 63/416,529, filed on Oct. 15, 2022.

(51) Int. Cl. A61K 47/69; A61K 35/22

(52) U.S. Cl. CPC A61K 47/6901; A61K 35/22

(57) ABSTRACT

Extracellular vesicles (EVs) are natural liposome-like vesicles secreted by cells into the extracellular space. Provided herein are techniques to enrich cargo-loaded EVs over non-loaded EVs and contaminants. To achieve EVs were engineered to display their surface an antigenic tag for fast and efficient EV isolation by immunocapture and a fluorescent protein in the internal space of the EV. Cargo was loaded into the lumen of the EVs by fusing the cargo with an antibody or nanobody that has an affinity for the fluorescent protein of the internal space. To prevent potential antigenicity of the EVs, a TEV cleavage site was included allowing the removal of exposed antigenic tag from immunocaptured EVs, while preserving their luminal cargo.

### CLAIM OF PRIORITY

This application claims the benefit of U.S. Provisional Application Ser. No. 63/416,529, filed on Oct. 15, 2022. The entire contents of the foregoing are incorporated herein by reference.

### FEDERALLY SPONSORED RESEARCH OR DEVELOPMENT

This invention was made with Government support under Grant No. CA232103 awarded by the National Institutes of Health. The Government has certain rights in the invention.

### SEQUENCE LISTING

This application contains a Sequence Listing that has been submitted electronically as an XML file named “29539-0711WO1_ST26.XML.” The XML file, created on Oct. 12, 2023, is 15,520 bytes in size. The material in the XML file is hereby incorporated by reference in its entirety.

### TECHNICAL FIELD

This disclosure describes extracellular vesicle compositions as a vehicle for drug delivery.

### BACKGROUND

Recently, there has been a quest for carriers for large proteins, particularly in the context of gene editing applications. Despite the development of improved compact editors with robust activities for a wide range of target recognition sites with significantly reduced off-target effects, traditional non-integrating vectors like AAV or nanoparticles containing synthetic mRNA still encounter challenges in gene editing. These challenges include extended-expression times and limited encoding capacity. The latter constraint often requires the delivery of multiple different vectors to the same recipient, which reduces the therapeutic use of base and prime editing for precise modifications, including insertions, deletions, and substitutions in the genome.

### SUMMARY

Provided herein are fusion protein constructs which include an antigenic peptide sequence linked to one or more transmembrane domains, a protease cleavage motif disposed between the antigenic peptide and the one or more transmembrane domains, and a fluorescent protein, wherein when the fusion protein is expressed in a cell or inserted into the membrane of an extracellular vesicle (EV), the antigenic peptide is displayed on the outside of the plasma membrane and the fluorescent protein is present in the internal space of the EV. In some embodiments, the antigenic peptide sequence is selected from the group consisting of an ALFA-tag sequence, an AviTag sequence, a C-tag sequence, a Calmodulin-tag sequence, an intein Capture Tag sequence, a polyglutamate tag sequence, a polyarginine tag sequence, an E-tag sequence, a FLAG-tag sequence, a 3×FLAG-tag sequence, an HA-tag sequence, a His-tag sequence, a Myc-tag sequence, a NE-tag sequence, a Rho1D4-tag sequence, a S-tag sequence, a SBP-tag sequence, a Softag 1 sequence, a Softag3 sequence, a Spot-tag sequence, a Strep-tag sequence, a T7-tag sequence, a TC tag sequence, a Ty tag sequence, a V5 tag sequence, a VSV-tag sequence, a Xpress tag sequence, an isopeptag sequence, a SpyTag sequence, a SnoopTag sequence, a DogTag sequence, a SdyTag sequence, a BCCP sequence, a glutathione-S-transferase-tag sequence, a green-fluorescent protein tag sequence, a HaloTag sequence, a SNAP-tag sequence, a CLIP-tag sequence, a HUH-tag sequence, a maltose binding protein-tag sequence, a Nus-tag sequence, a Thioredoxin-tag sequence, and a Fc-tag sequence. In some embodiments, the protease cleavage motif is selected from the group consisting of a Tobacco Etch Virus (TEV) motif, a PreScission motif, a Thrombin motif, an Xa motif, an Enterokinase motif, a Carboxypeptidase A motif, a Carboxypeptidase B motif, a Carboxypeptidase P motif, a Carboxypeptidase Y motif, a Trypsin motif, a Pepsin motif, an Elastase motif, a Papain motif, a Proteinase K motif, a Subtilisin motif, a Chymotrypsin A4 motif, a Thermolysin motif, a DAPase/Cathepsin C motif, an Endoproteinase Arg-C motif, an Endoproteinase Glu-C motif, an Endoproteinase Lys-C motif, an Endoproteinase Asp-N motif, an Acylamino-acid releasing enzyme motif, a Pyroglutamate Aminopeptidase motif, and/or a matrix metalloproteinase motif. In some embodiments, the fluorescent protein is selected from the group consisting of mCherry, a green fluorescent protein (GFP), a yellow fluorescent protein (YFP), a blue fluorescent protein (BFP), a cyan fluorescent protein (CFP), an orange fluorescent protein (OFP), and a red fluorescent protein (RFP), or a luminescent protein. In some embodiments, the fluorescent protein comprises mCherry. In some embodiments, the transmembrane domain comprises tetraspanin CD63.

Provided herein are methods of generating a population of extracellular vesicles enriched for a hypoimmunogenic fusion protein construct of at least one transmembrane domain, and a fluorescent protein and a cargo of interest, wherein the method includes expressing a fusion protein construct, optionally filtering or concentrating the cell media, incubating the cell media with antibody-coated surface, wherein the antibody of the antibody-coated surface is capable of binding the antigenic peptide of the fusion protein construct, after incubating the cell media with antibody-coated surface, incubating the antibody-coated surface with a protease, wherein the protease cleaves the antigenic peptide from the fusion protein construct, thereby generating the population of extracellular vesicles enriched for the hypoimmunogenic fusion protein construct comprising at least one transmembrane domain, and a fluorescent protein, and a cargo of interest. In some embodiments, the method further includes introducing one or more vectors to a cell in cell media, wherein the one or more vectors express a fusion protein construct and a cargo of interest, wherein the fusion protein construct comprises an antigenic peptide sequence linked to one or more transmembrane domains, a protease cleavage motif disposed between the antigenic peptide sequence and at least one of the one or more transmembrane domains, and a fluorescent protein. In some embodiments, the fluorescent protein comprises mCherry. In some embodiments, the antigenic peptide sequence comprises a FLAG tag, optionally a 3×FLAG tag. In some embodiments, the protease cleavage motif comprises a Tobacco Etch Virus (TEV) motif. In some embodiments, the antibody-coated beads comprise an anti-FLAG antibody. In some embodiments, the protease comprises Tobacco Etch Virus (TEV). In some embodiments, filtering the cell media comprises using size-exclusion chromatography (SEC). In some embodiments, the cargo is linked to an antibody or antibody fragment. In some embodiments, the cargo is linked to a nanobody.

Provided herein are methods of delivering a cargo to a cell which include administering to a subject in need thereof a therapeutically effective amount of a population of extracellular vesicles enriched for a hypoimmunogenic fusion protein construct. In some embodiments, the administering is intravenous administration. In some embodiments, the methods further comprise an antibody linked to a cargo of interest. In some embodiments, the antibody is a nanobody. In some embodiments, the cargo of interest is an enzyme selected from the group consisting of a site-specific recombinase, a transposase, a prime editor, a base editor, a Fanzor, a transcription activator-like effector nuclease (TALEN), or a zinc-finger nuclease (ZFN). In some embodiments, the enzyme comprises Cre recombinase. In some embodiments, the cargo of interest is a CRISPR/Cas component. In some embodiments, the CRISPR/Cas component is selected from SpCas9, SaCas9, StCas9, NmCas9, FnCas9, CjCas9, ScCas9, SpCas9-HF1, eSpCas9, HypaCas9, Fok1-Fused dCas9, xCas9, SpRY/SpG, dCas9Cas4, Cas1, Cas2, Cas3, CasX, CasY, Cas12a/Cpf1, Cas12b, Cas14a, Cas13a, Cas13b, Cas13d, and base editors, wherein the CRISPR/Cas component can be complexed with an appropriate guide RNA.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A shows constructs encoding anti-mCherry nanobody (Nb) fused payloads. Multiple protein cargos, including eGFP, CRE recombinase, and KKH-SaCas9 were fused to an anti-mCherry Nb.

FIG. 1B shows control constructs encoding non-specific Nb fused payloads. Transgene variants were generated with eGFP and CRE recombinase fused to a Nb that does not target mCherry.

FIG. 1C shows general strategy to apply the E-NoMi-Red platform (Nanoluciferase outside-mCherry inside (NoMi)) for the enrichment of cargo-loaded NoMi-Red EVs. Plasmid vector encoding Nb-fused cargo was transfected to E-NoMi-Red construct expressing cells to secrete cargo-loaded NoMi-Red EVs. A cargo of interest fused to anti-mCherry Nb that has affinity for mCherry that faces the cytosol of the membrane-embedded NoMi-Red protein in E-NoMi-Red expressing cells. As a consequence, EVs that contain NoMi-Red will adopt the Nb-fused cargo. Subsequently, cargo-loaded NoMi-Red EVs were isolated using a one-step procedure with bead-based FLAG-tag immunocapture, which effectively eliminates contaminants like free proteins and EVs without cargo loading. Of note, the term “E-NoMi-Red” refers to the genetic construct, where “E” denotes the EF-1alpha promoter, and the term “NoMi-Red” will be used for the recombinant protein found in the EVs.

FIG. 1D shows relative mCherry fluorescence intensity (RFI) per CD63 positive EV generated with two isolation methods, FLAG affinity immunocapture and SEC. EVs were attached to anti-CD63 spot of the Exoview™ chip and were visualized using a fluorescently tagged anti-CD63 antibody and mCherry fluorescence derived from luminal part of E-NoMi-Red. Exoview™ data represent mean with SD (error bars) of three different Exoview™ spot.

FIG. 1E shows relative GFP fluorescence intensity (RFI) per CD63/mCherry double positive EV from two different Nb-fused GFP-loaded EVs (with and without specific Nb) after immunocapture. EVs bound to the Exoview™ chip were visualized using anti-CD63 antibody, mCherry from luminal part of E-NoMi-Red, and eGFP from Nb-fused GFP. Exoview™ data represent mean with SD (error bars) of three different Exoview™ spot.

FIG. 1F shows gating strategy for Cre reporter cells with FlowJo software used to select far-red positive cells post-EV-delivery of Cre. In black, the negative control (only reporter cells), and in red the positive control (transfected with Cre plasmid) is shown.

FIG. 1G shows flow cytometry of Cre-reporter cells transfected with Cre-loaded NoMi-Red EVs. Percentage of far-red positive cells was plotted against Nanoluc signal, which is an indicator of the NoMi-Red protein quantity in the EV sample. Cell population data of positive and negative control were shown as dotted line.

FIG. 1H shows RT-qPCR of cDNA extracted from Cre-reporter cells transfected with Cre-loaded E-NoMi-Red EVs. CT values of floxed reporter were plotted against NanoLuc signal. CT value from positive and negative control were shown as dotted line.

FIG. 1I shows E-‘No’Mi-Red-expressing cell suspension cultures were exposed to TEV protease at room temperature or 37° C. Bioluminescence from NanoLuc in the extracted medium was measured at multiple time points (0 h, 2 h, 4 h, 6 h, 24 h). Data represent the mean (line) of three different samples with each value (symbol).

FIG. 2A shows isolation of NoMi-Red EVs through size-exclusion chromatography (SEC) and anti-FLAG tag immunocapture. Upconcentrated conditioned media from E-NoMi-Red expressing cells were collected and split into 2 tubes. Media from one tube went through SEC, followed by measurement of Nanoluc signal in each fraction. SEC fractions 5 to 10, showing high Nanoluc signal from NoMi-Red on EVs surface, were collected as EVs. On the other hand, anti-FLAG affinity beads were added to media from another tube. Nanoluc signals on FLAG beads, and in suspension were measured and illustrated as a percentage of total bioluminescence. EVs on anti-FLAG tag beads were then eluted by incubation with 3×FLAG peptide. EVs isolated through two different methods were applied to nanoparticle tracking analysis (NTA). NTA data represents the mean with SD (error bars) of three measurements.

FIG. 2B shows levels of NoMi-Red positive EVs in cell media-derived suspensions isolated with immunocapture or SEC. Bars representing mCherry-positive EVs captured by anti-CD63 tethered antibody printed on an Exoview™ chip. Data were shown as a percentage of all CD63-positive EVs discriminated based on anti-CD63 antibody.

FIG. 2C shows NoMi-Red positive EVs were isolated with immunocapture from E-NoMi-Red expressing cells with expression of Nb-fused eGFP that does not have mCherry/NoMi-Red affinity (left bar) or has mCherry/NoMi-Red affinity and thus in EVs (right bar). Based on Exoview™ chip countings (see B), a similar percentage of NoMi-Red EVs were isolated from the cell media.

FIG. 2D shows Nb-fused GFP loading in E-NoMi Red EVs was dissimilar in the two conditions. The bars represent the percentage of CD63/mCherry/GFP-triple-positive EVs in each condition. Exoview™ data in (B) to (D) represent the mean with SD (error bars) of three different Exoview™ spots.

FIG. 3A shows determination of cargo loading into NoMi-Red EVs. NoMi-Red EVs isolated with immunocapture from media derived from E-NoMi-Red-expressing cells, both with and without cargo were applied on western blotting. NoMi-Red band (77.9 kDa) was detected in all three immunocaptured EV samples to a similar extent by immunoblot against FLAG-tag. Immunoblot against Cre showed Cre protein only in NoMi-EVs isolated from media of E-NoMi-Red and specific Nb double-expressing cells. On the right is a cartoon demonstrating all the components in our EVs and the predicted molecular weights in kDa.

FIG. 3B shows illustration describing Cre reporter cells that undergo a color change from blue to far-red fluorescence upon successful Cre delivery. This model is used to test Cre-loading into EVs.

FIG. 3C shows Cre-reporter cells transfected with increasing doses of specific Nb-based Cre-loaded NoMi-Red EVs (ranging from 2.8×10\hat{ }9 to 1.3×10\hat{ }6 EVs as estimated by NTA) were evaluated through immunohistochemistry (IHC). Reporter cells were transfected with Cre plasmid DNA as positive control (PC), and cells without Cre-loaded NoMi-Red EVs as negative control (NC). Scale bar, 100 μm.

FIG. 3D shows flow cytometry of Cre-reporter cells transfected with Cre-loaded NoMi-Red EVs. Histograms of far-red fluorescence from floxed reporter cells in different doses of Cre-loaded NoMi-Red EVs were shown as overlays (left). The percentage of far-red positive cells was plotted against a dose of EVs (right). Positive cell population data from positive (Cre-encoding plasmid transfection) and negative control (only reporter cells) were shown as dotted lines.

FIG. 3E shows RT-qPCR of cDNA extracted from Cre-reporter cells transfected with Cre-loaded NoMi-Red EVs. Gel running of qPCR products showed the presence of floxed-specific amplicon around 300 bp (left). CT values of floxed reporter were plotted against a dose of EVs (right). CT values from positive (Cre-encoding plasmid transfection) and negative control (only reporter cells) were shown as dotted lines.

FIG. 3F shows Cre-reporter cells transfected with specific Nb-based Cre-loaded NoMi-Red EVs isolated with two different methods, FLAG immunoaffinity capture or SEC, were evaluated through immunohistochemistry (IHC). 8.9×10\hat{ }9 EVs and 8.9×10\hat{ }8 EVs were transfected into reporter cells. Scale bar, 100 μm.

FIG. 3G shows cargo loading of SaCas9 fused to anti-mCherry Nb. SaCas9-reporter cells transfected with increasing doses of SaCas9-loaded NoMi-Red EVs (ranging from 4.0×10\hat{ }10 to 5.5×10\hat{ }7 EVs as estimated by NTA) were evaluated through NanoLuc signal in media. Reporter cells were transfected with SaCas9 plasmid DNA as positive control (PC), and cells without SaCas9-loaded E-NoMi-Red EVs as negative control (NC).

FIG. 4A shows constructs encoding E-NoMi-Red and E-‘No’Mi-Red. The term E-‘No’Mi-Red is noted with ′ representing the position of the TEV cleavage sites. E-‘No’Mi-Red was generated with two Tobacco Etch Virus (TEV) protease cleavage sites flanking the 3×FLAG tag and Nanoluc-encoded sequence.

FIG. 4B shows illustration describing the removal of 3×Flag tag and NanoLuc from E-‘No’Mi-Red with TEV protease. TEV protease cleavage sites are sensitive to TEV protease activity, allowing for the release of a 24 kDa loop from the surface of our ‘No’Mi-Red EVs.

FIG. 4C shows general strategy to apply the E-‘No’Mi-Red platform for ‘No’Mi-Red EV isolation after loading. The 3×FLAG tag can still be utilized to select cargo-loaded ‘No’Mi-Red EVs, but the release of cargo-loaded EVs after immunocapture will be achieved by adding TEV protease instead of a FLAG tag elution peptide. The 24 kDa loop will be retained by the immunocapture beads to prevent the presence of antigenic 3×FLAG tag in our final EV sample, which can be assessed through Nanoluc luminescence on FLAG affinity beads.

FIG. 5A shows E-‘No’Mi-Red-expressing cell suspension cultures were exposed to TEV protease at 4° C. Bioluminescence from NanoLuc in extracted medium was measured at multiple time points (0 h, 1 h, 2 h, 4 h, 6 h, 24 h).

FIG. 5B Detection of 24 kDa loop from the cell surface through western blot. Extracted medium in (A) was validated with western blotting against FLAG-tag. Immunoblot showed a 24 kDa loop in the TEV+ sample, but not in the TEV− sample. On the other hand, a gradual increase in anti-FLAG antibody signal at 78 kDa was observed in the TEV-sample, which is the predicted size of the uncleaved ‘No’Mi-Red protein. Immunoblot against CD81, a common EV marker, showed an increase of CD81 over time in both TEV+ and TEV− samples.

FIG. 5C shows NanoLuc signal in each SEC fraction from condition media.

FIG. 5D shows NanoLuc signal in each SEC fraction from FLAG-tag elution sample. The majority of the Nanoluc signal was observed in the initial SEC fractions representing EVs, while the later SEC fractions, representing smaller non-EV particulates, displayed minimal signal.

FIG. 5E shows FLAG-tag immunocaptured ‘No’Mi-Red EVs were treated with TEV protease, followed by measurement of bioluminescence from NanoLuc. The majority of NanoLuc signal was detected on FLAG-tag beads, and almost no Nanoluc signal was detected in suspension, indicating almost all the antigenic 24 kDa loops were retained on the beads in the TEV+ sample and all the EVs remained on the beads of the TEV-sample.

FIG. 5F Detection of 24 kDa loop from the EV samples incubated with TEV protease through western blot. Immunoblot against FLAG-tag showed 24 kDa loop in the TEV+ sample, but not in the TEV− sample.

### DETAILED DESCRIPTION

Extracellular vesicles (EVs) are naturally secreted membrane-enclosed vesicles containing a variety of cell-adopted biomolecules. From a delivery vector point-of-view, they hold the potential to address a current gap in the field for delivery of proteins to recipient target cells (Breyne et al., 2022). For gene editing purposes, recombinant editors within EVs could enter the cytoplasm, navigate to the cell nucleus, induce edits in the genome, and rapidly degrade to minimize the risk of off-target effects. Although EVs have been suggested as a promising carrier for premade therapeutic payloads that can be generated by engineered cells in culture, several challenges still remain: (1) different subtypes of EVs carry distinct sets of biomolecules; (2) EVs primarily transport small or fragmented cargo (O'Brien et al., 2020); (3) purifying EVs is complicated due to the presence of free proteins and other biomolecules; (4) competition with the “natural” cargo of EVs can hinder targeted loading efforts; (5) cargo-loaded EVs are diluted among non-cargo-loaded ones; (6) determining the cargo concentration within EVs can be challenging; (7) higher doses of EV-loaded proteins are often needed compared to DNA or mRNA delivery, which can generate multiple proteins upon delivery. Described herein are compositions that are enriched for cargo-loaded EVs, and methods of making them.

### Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, the term “about” or “approximately” in the context of a numerical value or range means ±10% of the numerical value or range recited or claimed, unless otherwise noted.

As used herein, the term “extracellular vesicle” or “EV” refers to a cell-derived vesicle comprising a membrane that encloses an internal space. In some embodiments, the EV has a plasma membrane. Extracellular vesicles comprise all membrane-bound vesicles that have a smaller diameter than the cell from which they are derived. Generally extracellular vesicles range in diameter from 20 nm to 1000 nm, and can comprise various macromolecular cargo within the internal space. The cargo can comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

An “extracellular vesicle (EV)” refers to a cell-derived vesicles of 20 nm up to 2 μm comprising a membrane that encloses an internal space that are generated from cells. Exosomes are a species of extracellular vesicle that are between 20-300 nm in diameter, for example 40-200 nm in diameter. EVs and exosomes comprise lipids, fatty acids, and polypeptides from the cell, and optionally comprise cargo (e.g., a therapeutic agent), a polynucleotide (e.g., a nucleic acid, RNA, or DNA), a sugar (e.g., a simple sugar, polysaccharide, or glycan) or other molecules. The EVs and exosomes can be derived from a producer cell, and isolated from the producer cell based on its size, density, biochemical parameters, or a combination thereof.

As used herein the term “producer cell” refers to a cell used for generating an extracellular vesicle or exosome. A producer cell can be a cell cultured in vitro, or a cell in vivo. A producer cell includes, but is not limited to, a cell known to be effective in generating exosomes, e.g., HEK293 cells, Chinese hamster ovary (CHO) cells, and mesenchymal stem cells (MSCs). Other mammalian cells can also be used, including cultured mammalian (e.g., human) cell lines and primary cells. A producer cell can be grown or cultured in any appropriate cell media.

As used herein, “operably linked” indicates when two or more sequences (e.g., nucleotide sequence or amino acid sequence) are linked by functional linkage. For example, a promoter is operably linked to a nucleic acid sequence encoding a protein or RNA in order to affect the expression of the coding sequence. The functional linkage with an expression vector can be achieved by the recombinant DNA technology well known to those in the art, and particularly the site-specific DNA cleavage and linkage can be achieved by using the conventional enzyme well known to those in the art.

As used herein, the terms “isolate,” “purify,” “extracted,” and “enriched” can refer to the state of a preparation (e.g., a plurality of known or unknown amount and/or concentration) of desired EVs, that have undergone one or more processes of purification, e.g., a selection or an enrichment of the desired EV preparation. In some embodiments, isolating or purifying as used herein is the process of removing, partially removing (e.g., a fraction) of the EVs from a sample containing producer cells. In some embodiments, an isolated EV composition has no detectable undesired activity or, alternatively, the level or amount of the undesired activity is at or below an acceptable level or amount. In other embodiments, an enriched EV preparation has an amount and/or concentration of desired EVs at or above an acceptable amount and/or concentration. In other embodiments, the isolated EV composition is enriched as compared to the starting material (e.g., producer cell preparations) from which the composition is obtained. This enrichment can be by 10%, 20%, 30%, 40%, 50%, 60%, 70%, 80%, 90%, or 95% as compared to the starting material. In some embodiments, the methods herein generate enriched populations of EVs that comprise at least 10%, 20%, 30%, 40%, 50%, 60%, 70%, 80%, 90%, or 95% cargo-loaded EVs.

Preferably, the methods herein generate EV populations that are substantially free of residual biological products. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

By “reference” is meant a standard or control condition.

The terms “subject,” “patient,” “individual,” and the like as used herein are not intended to be limiting and can be generally interchanged. The subject is a mammal, e.g., a human, a primate, a mouse, a rat, a dog, a cat, a horse, as well as livestock or animals grown for food consumption, e.g., cattle, sheep, pigs, chickens, and goats. In some embodiments, the mammal is a human.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

### Fusion Protein Construct

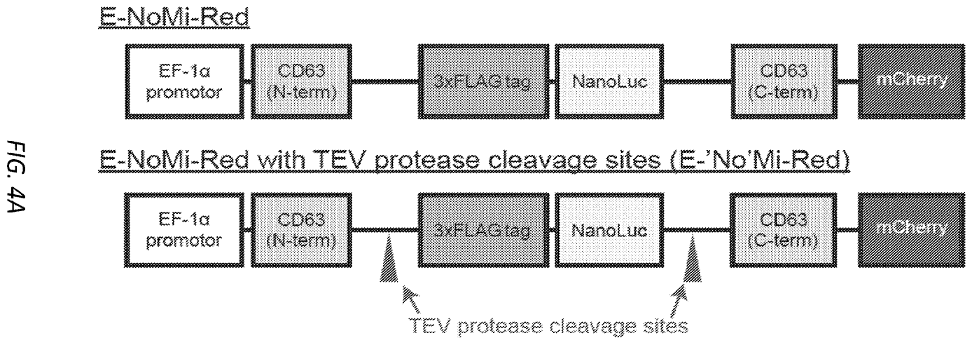

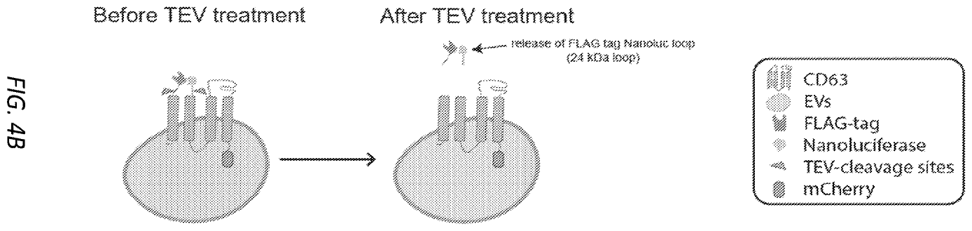

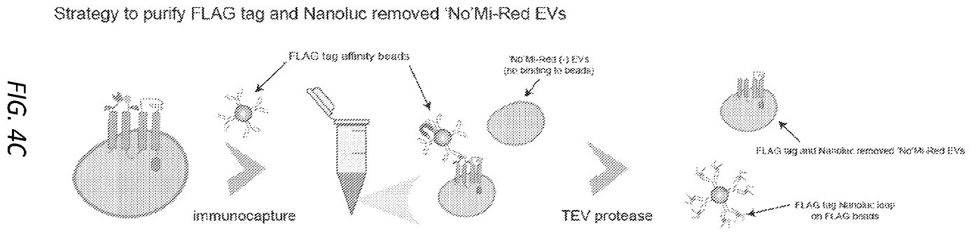

Provided herein are fusion protein constructs and fusion protein constructs that have been made hypoimmunogenic via the methods disclosed herein. As used herein, “hypoimmunogenic” refers to a fusion protein construct that evokes a lower immunogenic response than a fusion protein construct that has not been made hypoimmunogenic via the methods disclosed herein. The fusion proteins are illustrated in FIG. 4B and include an antigenic peptide: at least one transmembrane domain: a protease cleavage site between the antigenic peptide and the at least one transmembrane domain; and a fluorescent protein. When the fusion protein is expressed in a cell or inserted into the membrane of an extracellular vesicle (EV), the antigenic peptide is displayed on the outside of the plasma membrane and the fluorescent protein is present in the internal space of the EV, and the antigenic peptide can be cleaved completely off by the protease acting at the cleavage site.

The fusion protein constructs can include transmembrane proteins or single transmembrane domains therefrom. The transmembrane proteins can include single-pass transmembrane proteins and multipass transmembrane proteins. Single pass transmembrane proteins typically include an extracellular domain, a transmembrane domain, and an intracellular domain. Any suitable single pass transmembrane protein can be used to construct the fusion protein construct, including any of the single pass transmembrane proteins identified on Uniprot (uniprot.org/locations/SL-9906), or membranome (membranome.org), each of which is incorporated by reference in its entirety.

Multipass/multiple transmembrane domain proteins also typically include an extracellular domain, two or more transmembrane domains and an intracellular domain. Any suitable multiple transmembrane domain protein can be used to construct the fusion protein construct, including any of the multiple transmembrane proteins identified by Uniprot (uniprot.org/locations/SL-9909), which is incorporated by reference in its entirety.

Tetraspanins are a family of multipass membrane proteins found in all multicellular eukaryotes, and also referred to as the transmembrane 4 superfamily (TM4SF) proteins. They have four transmembrane alpha-helices and two extracellular domains, one short extracellular domain or loop, and one longer extracellular domain/loop. Although several protein families have four transmembrane alpha-helices, tetraspanins are defined by conserved amino acid sequences including four or more cysteine residues in the EC2 domain, with two in a highly conserved ‘CCG’ motif. Examples of tetraspanin proteins (and genes) that can be used to generate a hypoimmunogenic fusion protein construct can include TSPAN1 (TSPAN1), TSPAN2 (TSPAN2), TSPAN3 (TSPAN3), TSPAN4 (TSPAN4), TSPAN5 (TSPAN5), TSPAN6 (TSPAN6), TSPAN7 (TSPAN7), TSPAN8 (TSPAN8), TSPAN9 (TSPAN9), TSPAN10 (TSPAN10), TSPAN11 (TSPAN11), TSPAN12 (TSPAN12), TSPAN13 (TSPAN13), TSPAN14 (TSPAN14), TSPAN15 (TSPAN15), TSPAN16 (TSPAN16), TSPAN17 (TSPAN17), TSPAN18 (TSPAN18), TSPAN19 (TSPAN19), TSPAN20 (UPK1B), TSPAN21 (TSPAN21), TSPAN22 (PRPH2), TSPAN23 (TSPAN23), TSPAN24 (CD51), TSPAN25 (CD53), TSPAN26 (CD37), TSPAN27 (CD82), TSPAN28 (CD81), TSPAN29 (CD9), TSPAN30 (CD63), TSPAN31 (TSPAN31), TSPAN32 (TSPAN32), and TSPAN33 (TSPAN33).

For example, CD63, CD9, CD81 can be used to generate a hypoimmunogenic fusion protein using CD63 (murine) NP_001269895.1: CD63 (human) NP_001771.1: CD9 NP_001760.1: CD81 NP_004347.1: CD82 NP_002222.1. Additional tetraspanins that can be used in fusion proteins include those described in Kummer D, et al. Tetraspanins: integrating cell surface receptors to functional microdomains in homeostasis and disease. Med Microbiol Immunol. 2020 August: 209 (4): 397-405 and Prashant Kesharwani, Nanotechnology-Based Targeted Drug Delivery Systems for Lung Cancer, Academic Press, 2019, Pages 39-75, ISBN 9780128157206, doi.org/10.1016/B978-0-12-815720-6.00003-4. As described herein, tetraspanins have been engineered to carry an antigenic peptide sequence, at least one protease cleavage motif disposed between the antigenic peptide and at least one of the one or more transmembrane domains and a fluorescent protein.

The fusion protein constructs described herein can include an antigenic peptide. Non-limiting examples of antigenic peptides include FLAG-tag, ALFA-tag, AviTag, C-tag, Calmodulin-tag, intein Capture Tag, polyglutamate tag, polyarginine tag, E-tag, 3×FLAG-tag, HA-tag, His-tag, Myc-tag, NE-tag, Rho1D4-tag, S-tag, SBP-tag, Softag 1, Softag3, Spot-tag, Strep-tag, T7-tag, TC tag, Ty tag, V5 tag, VSV-tag, Xpress tag, isopeptag, Spy Tag, SnoopTag, DogTag, SdyTag, BCCP, glutathione-S-trasferase-tag, green-fluorescent protein tag, HaloTag, SNAP-tag, CLIP-tag, HUH-tag, maltose binding protein-tag, Nus-tag, Thioredoxin-tag, and Fc-tag. Extracellular vesicles comprising hypoimmunogenic fusion protein constructs do not include an antigenic peptide.

The fusion protein constructs described herein can include at least one protease cleavage motif. Non-limiting examples of protease cleavage motifs include motifs that can be cleaved by Tobacco Etch Virus (TEV), PreScission, Thrombin, Xa, Enterokinase, Carboxypeptidase A, Carboxypeptidase B, Carboxypeptidase P, Carboxypeptidase Y, Trypsin, Pepsin, Elastase, Papain, Proteinase K, Subtilisin, Chymotrypsin A4, Thermolysin, DAPase/Cathepsin C, Endoproteinase Arg-C, Endoproteinase Glu-C, Endoproteinase Lys-C, Endoproteinase Asp-N, Acylamino-acid releasing enzyme, pyroglutamate aminopeptidase, and matrix metalloproteinases (MMPs) as discussed in Cieplak P, and Strongin A Y. Matrix metalloproteinases—From the cleavage data to the prediction tools and beyond. Biochim Biophys Acta Mol Cell Res. 2017 November; 1864(11 Pt A):1952-1963. doi: 10.1016/j.bbamcr.2017.03.010, and any of the enzymes/recognition sites disclosed in Waugh D S. An overview of enzymatic reagents for the removal of affinity tags. Protein Expr Purif. 2011 December; 80(2):283-93. doi: 10.1016/j.pep.2011.08.005, each of which is incorporated by reference in its entirety.

The fusion protein constructs described herein can include a fluorescent protein. In some embodiments, the fluorescent protein is selected from the group consisting of green fluorescent proteins (GFPs), yellow fluorescent proteins (YFPs), blue fluorescent proteins (BFPs), cyan fluorescent proteins (CFPs), orange fluorescent proteins (OFPs), and red fluorescent proteins (RFPs). Some non-limiting examples of green fluorescent proteins (GFPs) include, EGFP, Emerald, Superfolder GFP, Azami Green, mWasabi, TagGFP, TurboGFP, AcGFP, ZsGreen, and T-Sapphire. Some non-limiting examples of yellow fluorescent proteins (YFPs) include, EYFP, Topaz, Venus, mCitrine, YPet, TagYFP, PhiYFP, ZsYellow1, and mBanana. Some non-limiting examples of blue fluorescent proteins (BFPs) include, EBFP, EBFP2, Azurite, and mTagBFP. Some non-limiting examples of cyan fluorescent proteins (CFPs) include, ECFP, mECFP, Cerulean, mTurquoise, CyPet, AmCyan1, Midori-Ishi Cyan, TagCFP, and mTFP1 (Teal). Some non-limiting examples of orange fluorescent proteins (OFPs) include, Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, and mTangerine. Some non-limiting examples of red fluorescent proteins (RFPs) include mRuby, mApple, mStrawberry, AsRed2, mRFP1, JRed, mCherry, HcRed1, mRaspberry, dKeima-Tandem, HcRed-Tandem, mPlum, and AQ143. Useful fluorescent proteins also include mutants and spectral variants of these proteins which retain the ability to fluoresce. See e.g., Shaner et al., Nat. Biotech. 22:1567 (2004), Tag-RFP (Shaner, N. C. et al., 2008 Nature Methods, 5(6), 545-551), each of which is incorporated by reference in its entirety.

The fluorescent protein of the fusion protein constructs described herein can include a luminescent protein. Examples of luminescent proteins include nano-lantern (including yellow nano-lantern, cyan nano-lantern, and orange nano-lantern as described in Takai A, et al, Expanded palette of nano-lanterns for real time multicolor luminescence imaging. PNAS, 112 (14) 4352-4356, doi.org/10.1073/pnas.1418468112), VRL 10.3, eBAF-Y, RLuc8_S257G, RLuc8, RLuc, or any of the luminescent proteins described in Saito K, Chang Y F, Horikawa K, Hatsugai N, Higuchi Y, Hashida M, Yoshida Y, Matsuda T, Arai Y, Nagai T. Luminescent proteins for high-speed single-cell and whole-body imaging. Nat Commun. 2012; 3:1262. doi: 10.1038/ncomms2248, and Vysotski E S. Bioluminescent and Fluorescent Proteins: Molecular Mechanisms and Modern Applications. Int J Mol Sci. 2022 Dec. 23; 24(1):281. doi: 10.3390/ijms24010281, each of which is incorporated by reference in its entirety.

As one example, provided herein are NoMi constructs for inclusion in an extracellular vesicle (EV). NoMi features a human CD63 tetraspanin scaffold with mCherry and Nanoluciferase (Nanoluc) reporters projecting onto the inner and outer surfaces of extracellular vesicles (EVs), respectively. To facilitate immunocapture of NoMi EVs from crude samples, we have incorporated a 3×FLAG tag on the outer surface. This is particularly useful for the isolation and recovery of rare EVs traveling from the brain to the blood (Maalouf et al., 2023). Although initially developed to assess diagnostic brain markers carried by EVs, the NoMi platform is used herein as a delivery system. Essentially, the luminal mCherry projection of NoMi is utilized as a tether for a mCherry-specific nanobody. This nanobody is fused with a cargo of interest, for example, eGFP, CRE recombinase, and saCas9. Alongside the versatility in terms of recombinant cargo, this loading technique exhibits effective transfer to recipient cells and outperformed EVs derived from conventional size-exclusion chromatography (SEC) EV isolation methods. Finally, two tobacco etch virus (TEV) sites are incorporated to enable the removal of the 3×FLAG tag and NLuc located on the exterior of NoMi, thereby preserving the low-immunogenic properties of the EVs.

### Cargo

The cells and/or extracellular vesicles described herein can carry a cargo of interest. The cargo of interest is fused to an antibody, antibody fragment, or nanobody capable of binding the fluorescent protein of the fusion protein construct. In some embodiments, a vector encoding the antibody, antibody fragment, or nanobody and cargo of interest is introduced to a producer cell, wherein the producer cell generates the antibody or nanobody-fused cargo. In some embodiments, the antibody or nanobody-fused cargo is within an extracellular vesicle, wherein the extracellular vesicle further comprises any of the fusion protein constructs described herein.

The cells and/or extracellular vesicles described herein can include a protein or nucleic acid, e.g., an enzyme as a cargo of interest. For example, an enzyme as a cargo of interest can include site-specific recombinase components (e.g., Flp recombinase, DK recombinase, B2 recombinase, B3 recombinase, R recombinase, Cre recombinase, VCre recombinase, SCre recombinase, Vika recombinase, Dre recombinase, λ-Int recombinase, HK022 recombinase, QC31 recombinase, BxB1 recombinase, Gin recombinase, and Tn3 recombinase) as described in Gaj T, Sirk S J, Barbas C F 3rd. Expanding the scope of site-specific recombinases for genetic and metabolic engineering. Biotechnol Bioeng. 2014 January; 111(1):1-15. doi: 10.1002/bit.25096 and Jelicic M, et al., Discovery and characterization of novel Cre-type tyrosine site-specific recombinases for advanced genome engineering, Nucleic Acids Research, Volume 51, Issue 10, 9 Jun. 2023, Pages 5285-5297, doi.org/10.1093/nar/gkad366: U.S. Pat. No. 7,566,814: U.S. Pat. No. 11,661,590: U.S. Pat. No. 11,030,531, each of which is incorporated by reference in its entirety.

Other examples of enzymes as a cargo of interest include transposases (e.g., sleeping beauty, piggyBac, Tol2, as discussed in Sandoval-Villegas N, Nurieva W, Amberger M, Ivics Z. Contemporary Transposon Tools: A Review and Guide through Mechanisms and Applications of Sleeping Beauty, piggy Bac and Tol2 for Genome Engineering. Int J Mol Sci. 2021 May 11; 22(10):5084. doi: 10.3390/ijms22105084, or Academ, Crypton (CryptonA, CryptonF, CryptonI, CryptonS, CryptonV), Dada, EnSpm/CACTA, Ginger1, Ginger2/TDD, Harbinger, hAT, Helitron, IS3EU, ISL2EU, Kolobok, Mariner/Tc1, Merlin, MuDR, Novosib, P, piggyBac, Polinton, Sola (Sola1, Sola2, Sola3), Transib, Zator, Zisupton, or any of the transposases discussed in Kojima K K. Human transposable elements in Repbase: genomic footprints from fish to humans. Mob DNA. 2018 Jan. 4; 9:2. doi: 10.1186/s13100-017-0107-y, or U.S. Pat. No. 10,233,454: U.S. Pat. No. 10,947,534: U.S. Pat. No. 11,485,959; US20220380758, each of which is incorporated by reference in its entirety.

The cells and/or extracellular vesicles described herein can include prime editor components (e.g., PE2, PE3, PE3b, PE4, and PE5) and any variants as described in Zhao et al. Prime editing: advances and therapeutic applications. Trends in Biotechnology. 2023, 41:8, P1000-1012 and Chen P J, et al. Enhanced prime editing systems by manipulating cellular determinants of editing outcomes, Cell, Volume 184, Issue 22, 2021, Pages 5635-5652.e29, doi.org/10.1016/j.cell.2021.09.018, WO2023015309, WO2020191242, and WO2023096977, each of which is incorporated by reference in its entirety.

The cells and/or extracellular vesicles described herein can include base editor components (e.g., BE1, BE2, HF2-BE2, BE3, HF,-BE3, YE1-BE3, EE-BE3, YEE-BE3, VQR-BE3, EQR-BE3, VRER-Be3, Sa-BE3, Sa-BE4, SaNe4-Gam, SaKKH-BE3, Cas12a-BE, Target-AID, xBE3, eA3A-BE3, BE-PLUS, TMA, CRISPR-X, ABE7.9, ABE7.10 ABE7.10*, xABE, ABESa, VQR-ABE, BRER-ABE, SaKKH-ABE) including any base editors described in Kantor A, McClements M E, MacLaren R E. CRISPR-Cas9 DNA Base-Editing and Prime-Editing. Int J Mol Sci. 2020 Aug. 28; 21(17):6240. doi: 10.3390/ijms21176240 and Rees H A, Liu D R. Base editing: precision chemistry on the genome and transcriptome of living cells. Nat Rev Genet. 2018 December; 19(12):770-788. doi: 10.1038/s41576-018-0059-1, and US20210198330: WO2020181180: WO2020181178, each of which is incorporated by reference in its entirety.

The cells and/or extracellular vesicles described herein can include Fanzor endonuclease components, for example any of the components described in Saito M, et al. Fanzor is a eukaryotic programmable RNA-guided endonuclease. Nature. 2023 August; 620(7974):660-668. doi: 10.1038/s41586-023-06356-2 and WO2023114872, which is incorporated by reference in its entirety.

The cells and/or extracellular vesicles described herein can include transcription activator-like effector nuclease (TALEN) components (e.g., Fok1-based, Pvul1-based, I-TevI-based, I-AniI-based I-OnuI-based, MutH-based), including any components or variants described in Sebastian Becker, Jens Boch, TALE and TALEN genome editing technologies, Gene and Genome Editing, Volume 2, 2021, doi.org/10.1016/j.ggedit.2021.100007, and Ser. No. 13/427,040 (U.S. Pat. No. 8,440,431); U.S. Ser. No. 13/427,137 (U.S. Pat. No. 8,440,432); and U.S. Ser. No. 13/738,381 (U.S. Pat. No. 8,697,853), each of which is incorporated by reference herein in its entirety.

The cells and/or extracellular vesicles described herein can include zinc-finger nuclease (ZFN) components, for example, any of the components described in Beerli et al. (2002) Nature Biotechnol. 20:135-141: Pabo et al. (2001) Ann. Rev. Biochem. 70:313-340: Isalan et al. (2001) Nature Biotechnol. 19:656-660: Segal et al. (2001) Curr. Opin. Biotechnol. 12:632-637: Choo et al. (2000) Curr. Opin. Struct. Biol. 10:411-416. Additionally the cells and/or extracellular vesicles can include ZFN described in and/or identified by the methods of US20150017728A1: U.S. Pat. Nos. 6,453,242; 6,534,261; 6,607,882; 5,789,538; 5,925,523; 6,007,988; 6,013,453; 6,410,248; 6,140,466; 6,200,759; and 6,242,568; as well as WO 98/37186: WO 98/53057: WO 00/27878: WO 01/88197, each of which is incorporated by reference herein in its entirety.

The cells and/or extracellular vesicles described herein can include a CRISPR/Cas component or complex as a cargo of interest. Non-limiting examples of CRISPR/Cas components include SpCas9, SaCas9, StCas9, NmCas9, FnCas9, CjCas9, ScCas9, SpCas9-HF1, eSpCas9, HypaCas9, Fok1-Fused dCas9, xCas9, SpRY/SpG, dCas9Cas4, Cas1, Cas2, Cas3, CasX, CasY, Cas12a/Cpf1, Cas12b, Cas14a, Cas13a, Cas13b, and Cas13d, as well as fusion proteins comprising the Cas, e.g., base editors. The Cas protein can be complexed with an appropriate guide RNA for that Cas, e.g., as a ribonucleoprotein complex. Exemplary CRISPR/Cas components can be found in WO2014204578, WO2015089465, WO2018170333, WO2018022634, and WO2019104058 each of which are incorporated by reference in its entirety.

### Vectors

Nucleic acids encoding a fusion protein construct or a therapeutically active fragment thereof can be incorporated into expression vectors for expression of a polynucleotide that encodes a fusion protein construct in a producer cell. Additionally, nucleic acids encoding an antibody-cargo construct can be incorporated into expression vectors. Expression constructs can include such components as promoters and can be administered in any effective carrier, e.g., any formulation or composition capable of effectively delivering the component gene to cells in vivo. Approaches include insertion of the gene in vectors such as plasmids or viral vectors, including lentiviral vectors. Viral vectors typically transduce cells directly.

Viral vectors capable of highly efficient transduction may be employed, including lentivirus or other suitable viral vectors, including any serotypes of AAV (e.g., AAV1-AAV12, AAV-9, and AAV-F) vectors, including recombinant or chimeric vectors. A typical approach for introduction of nucleic acid into a cell is by use of a viral vector containing nucleic acid, e.g., a cDNA encoding a fusion protein construct. Among other things, infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

A viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol. 158:97-129 (1992)). AAV vectors efficiently transduce various cell types and can produce long-term expression of transgenes in vivo. Although AAV vector genomes can persist within cells as episomes, vector integration has been observed (see for example Deyle and Russell, Curr Opin Mol Ther. 2009 August; 11(4): 442-447; Asokan et al., Mol Ther. 2012 April: 20(4): 699-708; Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989)). AAV vectors, such as AAV2, have been extensively used for gene augmentation or replacement and have shown therapeutic efficacy in a range of animal models as well as in the clinic: see, e.g., Mingozzi and High, Nature Reviews Genetics 12, 341-355 (2011): Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. Protocols for producing recombinant retroviruses and for infecting cells in vitro or in vivo with such viruses are known in the art, e.g., can be found in Ausubel, et al., eds., Current Protocols in Molecular Biology, Greene Publishing Associates, (1989), Sections 9.10-9.14, and other standard laboratory manuals.

Adenoviruses include over 50 serotypes (see, e.g., WO 95/27071, which is herein incorporated by reference). Adenoviruses are tractable through the application of techniques of molecular biology and may not require integration into the host cell genome. Recombinant Ad-derived vectors, including vectors that reduce the potential for recombination and generation of wild-type virus, have been constructed (see, e.g., international patent publications WO 95/00655 and WO 95/11984, which are herein incorporated by reference). In some instances, the AAV vector is selected from the group consisting of AAV1, AAV2, AAV3, AAV4, AAV5, AAV6, AAV7, AAV8, AAV9, AAVrh10, AAV11, and AAV12. In some embodiments, the AAV vector is AAV-9, AAV9-PHP.B, AAV-S, or AAV-F, see WO2020198737A1 herein incorporated by reference in its entirety. In some embodiments, the AAV vector is AAV-9 or AAV-F (see Beharry A, et al. The AAV9 Variant Capsid AAV-F Mediates Widespread Transgene Expression in Nonhuman Primate Spinal Cord After Intrathecal Administration. Hum Gene Ther. 2022 January: 33 (1-2): 61-75; see also Hanlon K S, et al., Selection of an Efficient AAV Vector for Robust CNS Transgene Expression, Molecular Therapy—Methods & Clinical Development, Volume 15, 2019, Pages 320-332). In some instances, a particular AAV serotype vector may be selected based upon the intended use, e.g., based upon the intended route of administration.

A vector as described herein can be a pseudotyped vector. Pseudotyping provides a mechanism for modulating a vector's target cell population. Pseudotyped vectors are those that contain the genome of one vector, in the capsid of a second vector. Additionally or alternatively, a “pseudotyped” lentivirus can be a lentiviral particle having one or more envelope glycoproteins that are encoded by a virus that is distinct from the lentiviral genome. The envelope glycoprotein may be modified, mutated, or engineered. Methods of pseudotyping are well known in the art. For instance, a vector may be pseudotyped with envelope glycoproteins derived from Rhabdovirus vesicular stomatitis virus (VSV) serotypes (Indiana and Chandipura strains), rabies virus (e.g., various Evelyn-Rokitnicki-Abelseth ERA strains and challenge virus standard (CVS)), Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV). A virus may be pseudotyped for transduction of one or more groups of cells. Without limitation, illustrative examples of pseudotyped vectors include recombinant AAV2/1, AAV2/2, AAV2/5, AAV2/6, AAV2/7, AAV2/8, AAV9, AAVrh10, AAV11, and AAV12 serotype vectors. It is known in the art that such vectors may be engineered to include a transgene encoding a human protein or other protein. Non-limiting examples of pseudotyped vectors include U.S. Pat. Nos. 9,803,218 and 10,993,999, each incorporated by reference in their entirety.

Various methods for application of AAV vector constructs in gene therapy are known in the art, including methods of modification, purification, and preparation for administration to human subjects (see, e.g., Viral Vectors for Gene Therapy: Methods and Protocols, ed. Machida, Humana Press, 2003). In addition, AAV based gene therapy targeted to cells of the CNS has been described (see, e.g., U.S. Pat. Nos. 6,180,613 and 6,503,888). High titer AAV preparations can be produced using techniques known in the art, e.g., as described in U.S. Pat. No. 5,658,776

A vector construct refers to a polynucleotide molecule including all or a portion of a viral genome and a transgene. In some instances, gene transfer can be mediated by a DNA viral vector, such as an adenovirus (Ad) or adeno-associated virus (AAV). Other vectors useful in methods of gene therapy are known in the art. For example, a construct as disclosed herein can include an alphavirus, herpesvirus, retrovirus, lentivirus, or vaccinia virus.

Non-native regulatory sequences, gene control sequences, promoters, non-coding sequences, introns, or coding sequences can be included in a nucleic acid as disclosed herein. The inclusion of nucleic acid tags or signaling sequences, or nucleic acids encoding protein tags or protein signaling sequences, is further contemplated herein. Typically, the coding region is operably linked with one or more regulatory nucleic acid components.

A promoter included in a nucleic acid as disclosed herein can be a tissue- or cell type-specific promoter, a promoter specific to multiple tissues or cell types, an organ-specific promoter, a promoter specific to multiple organs, a systemic or ubiquitous promoter, or a nearly systemic or ubiquitous promoter. A promoter can include any of the above characteristics or other promoter characteristics known in the art. Non-limiting promoters can include CMV, EF1a, SV40, CAG, PGK1, TRE, U6, Ubc, UAS, human beta actin, Ac5, polyhedrin, CaMKIIa, GAL1, GAL10, TEF1, GDS, ADHI, CaMV35S, Ubi, H1, GFAP, MPZ, U7, dox-inducible, and tet-inducible promoters.

In clinical settings, the gene delivery systems for the therapeutic gene can be introduced into a subject by any of a number of methods, each of which is known in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by intrathecal injection, by catheter or by stereotactic injection.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Methods of Generating a Population of Extracellular Vesicles (EVs) Enriched for a Cargo of Interest

The present methods and compositions can include EVs or a preparation thereof that contains one or more cargos, e.g., polypeptides or polynucleotides, as described herein, as well as producer cells expressing the fusion proteins described herein. The methods and compositions described herein can be applied to all EVs, a term which encompasses exosomes, shed microvesicles, oncosomes, ectosomes, and retroviral-like particles. The methods and compositions described herein can be applied to EVs of all sizes. In some embodiments, the EVs are between 50 and 150 nm. In some embodiments, the EVs are between 30 and 200 nm. In some embodiments the EVs are between 30 and 800 nm. In some embodiments, the EVS are as large as 2 μm. Such an EV or preparation is produced by the herein described methods.

As the term is used herein, an EV preparation refers to a population of EVs. Such a preparation is generated, for example, in vitro, by culturing cells expressing any fusion protein construct and cargo and isolating EVs produced by the cells. For example, vectors encoding for the fusion protein construct and the antibody-cargo construct can be introduced into a producer cell. The producer cell translates the vectors and packages the resulting constructs into extracellular vesicles. A population of extracellular vesicles enriched in the fusion protein construct and the cargo can be generated using methods that include an immunocapture step. For immunocapture, the cell media (optionally after being filtered, e.g., by passing the cell media through a size-exclusion chromatography column, and/or sorted to obtain EVs that comprise the intracellular fluorescent protein) is incubated with a surface (e.g., beads, plate, column, other surface) coated in anti-antigenic peptide antibodies. The extracellularly expressed antigenic peptide of the fusion protein construct binds the anti-antigenic antibody of the antibody-coated surface, capturing fusion protein construct expressing extracellular vesicles. The extracellular vesicle/antibody surface is incubated with a protease, wherein the protease cleaves the antigenic peptide from the fusion protein construct. The fusion protein construct-expressing extracellular vesicles without the antigenic peptide can then be collected, thereby generating a population of hypoimmunogenic fusion protein construct expressing extracellular vesicles.

Additional methods of isolating EVs are known in the art (Thery et al., Isolation and characterization of EVs from cell culture supernatants and biological fluids, in Current Protocols Cell Biology, Chapter 3, 322, (John Wiley, 2006); Palmisano et al., (Mol Cell Proteomics. 2012 August; 11(8):230-43) and Waldenström et al., ((2012) PLOS ONE 7(4): e34653.doi: 10.1371/journal.pone.0034653)), some examples of which are described herein: these methods can be used in conjunction with the fluorescent sorting and immunocapture step described herein. Such techniques for isolating EVs from cells in culture include, without limitation, sucrose gradient purification/separation and differential centrifugation, and can be adapted for use in a method or composition described herein. See, e.g., EP2010663B1. Other methods include size-exclusion chromatography, see WO2018112154, incorporated by reference in its entirety.

In some embodiments, the EVs are isolated by gentle centrifugation (e.g., at about 300 g) of the culture medium of the producer cells for a period of time adequate to separate cells from the medium (e.g., about 15 minutes). This leaves the EVs in the supernatant. In some embodiments, the culture medium or the supernatant from the gentle centrifugation, is more strongly centrifuged (e.g., at about 16,000 g) for a period of time adequate to precipitate cellular debris (e.g., about 30 minutes). This leaves the EVs in the supernatant. In some embodiments, the culture medium, the gentle centrifuged preparation, or the strongly centrifuged preparation is subjected to filtration (e.g., through a 0.22 μm filter or a 0.8 μm filter, whereby the EVs pass through the filter. In some embodiments, the filtrate is subjected to a final ultracentrifugation (e.g., at about 110,000 g) for a period of time that will adequately precipitate the EVs (e.g., for about 80 minutes). The resulting pellet contains the EVs and can be resuspended in a volume of buffer that yields a useful concentration for further use, to thereby yield the EV preparation. In some embodiments, the EV preparation is produced by sucrose density gradient purification.

The EV preparation includes a cargo. In some embodiments, an EV in a preparation will be a heterogeneous population, and each EV will contain a cargo that may or may not differ from that of other EVs in the preparation. The content of the cargos in an EV preparation can be expressed either quantitatively or qualitatively. One such method is to express the content as the percentage of total molecules within the EV preparation. By way of example, if the cargo is an mRNA, the content can be expressed as the percentage of total RNA content, or alternatively as the percentage of total mRNA content, of the EV preparation. Similarly, if the cargo is a protein, the content can be expressed as the percentage of total protein within the EV. In some embodiments, therapeutic EVs, or a preparation thereof, produced by the method described herein contain a detectable, statistically significantly increased amount of the cargo as compared to EVs not obtained using the methods described herein. In some embodiments, the cargo is present in an amount that is at least about 10%, 20%, 30% 40%, 50%, 60%, 70% 80% or 90%, more than in cargo obtained from control cells. Higher levels of enrichment may also be achieved. In some embodiments, the cargo is present in the EV population or preparation thereof, at least 2-fold more than control cell EV population. Higher fold enrichment may also be obtained (e.g., 3, 4, 5, 6, 7, 8, 9 or 10-fold).

In some embodiments, a relatively high percentage of the EV content is the cargo (e.g., achieved through overexpression or specific targeting of the molecule to EVs). In some embodiments, the EV content of the therapeutic molecule is at least about 10%, 20%, 30% 40%, 50%, 60%, 70% 80% or 90%, of the total (like) molecule content (e.g., the therapeutic molecule is an mRNA and is about 10% of the total mRNA content of the EV). Higher levels of enrichment may also be achieved. In some embodiments, the therapeutic molecule is present in the EV or preparation thereof, at least 2-fold more than all other such (like) molecules. Higher fold enrichment may also be obtained (e.g., 3, 4, 5, 6, 7, 8, 9 or 10-fold).

### Pharmaceutical Compositions and Methods of Administration

Compositions described herein include hypoimmunogenic fusion protein constructs and cells and/or EVs which express a hypoimmunogenic fusion protein construct and an antibody-fused cargo. In some embodiments, the composition includes any of the vectors disclosed herein for the expression of a hypoimmunogenic fusion protein construct and/or an antibody-fused cargo. In some embodiments, the pharmaceutical compositions of the disclosure can be delivered to a cell of interest for the purpose of providing the cell of interest with the cargo of the extracellular vesicle.

The methods described herein include pharmaceutical compositions comprising or consisting of a hypoimmunogenic fusion protein construct, as an active ingredient, and methods of use thereof. Pharmaceutical compositions typically include a pharmaceutically acceptable carrier. As used herein the language “pharmaceutically acceptable carrier” includes saline, solvents, dispersion media, coatings, isotonic and absorption delaying agents, and the like, compatible with pharmaceutical administration.

Pharmaceutical compositions are typically formulated to be compatible with its intended route of administration. In some embodiments, the pharmaceutical compositions are administered systemically. Examples of routes of administration include parenteral, e.g., intratumoral, intravenous, intradermal, subcutaneous, or intraperitoneal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid: buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate and gelatin. Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

The pharmaceutical compositions can be included in a kit, container, pack, or dispenser together with instructions for administration in a method described herein.

### Exemplary Sequences and Constructs

In some embodiments, the sequence of a protein or nucleic acid used in a composition or method described herein is at least 80%, 85%, 90%, 95%, 97%, 98%, or 99% identical to a reference sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

SEQ ID NO: 1 Anti-mCherry nanobody gBlock:
tTCGTGACCGCCGCCGGGATCACTCTCGGCATGGACGAGCTgTACAAGGA
GAACCTCTACTTTCAGTCCGGCGCGCCTATGGCCCAGGTGCAGCTGGTGG
AGAGCGGCGGCGGCCTGGTGCAGGCCGGCGGCAGCCTGAGACTGAGCTGC
GCCACCAGCGGCTTCACCTTCAGCGACTACGCCATGGGCTGGTTCAGACA
GGCCCCCGGCAAGGAGAGAGAGTTCGTGGCCGCCATCAGCTGGAGCGGCC
ACGTGACCGACTACGCCGACAGCGTGAAGGGCAGATTCACCATCAGCAGA
GACAACGTGAAGAACACCGTGTACCTGCAGATGAACAGCCTGAAGCCCGA
GGACACCGCCGTGTACAGCTGCGCCGCCGCCAAGAGCGGCACCTGGTGGT
ACCAGAGAAGCGAGAACGACTTCGGCAGCTGGGGCCAGGGCACCCAGGTG
ACCGTGAGCAAGGAGGCCATCTGATCGACAATCAACCTCTGGATTACAAA
ATTTGTGAAAGATT
SEQ ID NO: 2 anti-ALFA nanobody gBlock:
tTCGTGACCGCCGCCGGGATCACTCTCGGCATGGACGAGCTgTACAAGGA
GAACCTCTACTTTCAGTCCGGCGCGCCTGTGCAGCTGCAGGAGAGCGGCG
GCGGCCTGGTGCAGCCCGGCGGCAGCCTGAGACTGAGCTGCACCGCCAGC
GGCGTGACCATCAGCGCCCTGAACGCCATGGCCATGGGCTGGTACAGACA
GGCCCCCGGCGAGAGAAGAGTGATGGTGGCCGCCGTGAGCGAGAGAGGCA
ACGCCATGTACAGAGAGAGCGTGCAGGGCAGATTCACCGTGACCAGAGAC
TTCACCAACAAGATGGTGAGCCTGCAGATGGACAACCTGAAGCCCGAGGA
CACCGCCGTGTACTACTGCCACGTGCTGGAGGACAGAGTGGACAGCTTCC
ACGACTACTGGGGCCAGGGCACCCAGGTGACCGTGAGCAGCTCGACAATC
AACCTCTGGATTACAAAATTTGTGAAAGATT
SEQ ID NO: 3 TEV-containing loop gBlock:
GTGGGTGTCGGGGCACAGCTTGTCCTGAGTCAGACCATATCTAGAATTCC
TAGGGAGAACCTCTACTTTCAGTCCGACTACAAAGACCATGACGGTGATT
ATAAAGATCATGACATCGATTACAAGGATGACGATGACAAGGGTGGTTCT
GGTGGTGGTTCTGGTCGATCCACCATGGTCTTCACACTCGAAGATTTCGT
TGGGGACTGGCGACAGACAGCCGGCTACAACCTGGACCAAGTCCTTGAAC
AGGGAGGTGTGTCCAGTTTGTTTCAGAATCTCGGGGTGTCCGTAACTCCG
ATCCAAAGGATTGTCCTGAGCGGTGAAAATGGGCTGAAGATCGACATCCA
TGTCATCATCCCGTATGAAGGTCTGAGCGGCGACCAAATGGGCCAGATCG
AAAAAATTTTTAAGGTGGTGTACCCTGTGGATGATCATCACTTTAAGGTG
ATCCTGCACTATGGCACACTGGTAATCGACGGGGTTACGCCGAACATGAT
CGACTATTTCGGACGGCCGTATGAAGGCATCGCCGTGTTCGACGGCAAAA
AGATCACTGTAACAGGGACCCTGTGGAACGGCAACAAAATTATCGACGAG
CGCCTGATCAACCCCGACGGCTCCCTGCTGTTCCGAGTAACCATCAACGG
AGTGACCGGCTGGCGGCTGTGCGAACGCATTCTGGCGGAGAACCTCTACT
TTCAGTCCGGCGCGCCTTCTAGAATCCAGGGGGCTACCCCTGGCTCTCTG
TTGCCAGTGGTC
SEQ ID NO: 14 - TEV motif
ENLYFQS

### EXAMPLES

The invention is further described in the following examples, which do not limit the scope of the invention described in the claims.

### Example 1—Exemplary Methods

Gibson assembly to generate new constructs. To generate eGFP fused Nb constructs, a gblock (Integrated DNA Technologies, Inc.) of 514 bp encoding anti-mCherry nanobody was assembled into a SalI/BsrgI restricted vector (addgene #17446) containing eGFP. Similarly, an anti-alfa tag nanobody was assembled with a gblock of 481 bp into the SalI/BsrgI restricted vector and used as an aspecific mCherry-Nb. The amino acid sequence of anti-mCherry and anti-ALFA tag nanobodies was adopted from addgene plasmids (#162276 and #171818, respectively) after codon optimization for mammalian expression with SnapGene v7.0.2 software. We used these constructs to introduce CRE to generate CRE-Nb-expressing plasmids. Anti-mCherry-Nb expressing plasmid was restricted with BamHI/XcmI to introduce Cre that was amplified from an in-house plasmid with 5′-CACGCTGTTTTGACCTCCATAGAAGACACCGACTCTAGAGATGCCCAAGA AGAAGAGGAAGGT-3′ (SEQ ID NO: 4) and 5′-GGCCGCCGCCAGATTCCACCAGTTGCACCTGGGCCATGGGGGGGTGTATA TCGCCATCTTCCAGCAGG-3′ (SEQ ID NO: 5). Similarly, anti-ALFA tag Nb expressing plasmid was restricted with BamHI/Ascl to introduce Cre that was amplified from an in-house plasmid with 5′-CACGCTGTTTTGACCTCCATAGAAGACACCGACTCTAGAATGCCCAAGAA GAAGAGGAAGGT-3′ (SEQ ID NO: 6) and 5′-GCTGCACCAGGCCGCCGCCGCTCTCCTGCAGCTGCACAGGATCGCCATCT TCCAGCAGG-3′ (SEQ ID NO: 7). The KHH-SaCas9 was fused to anti-mCherry Nb by Gibson Assembly in Xhol/NotI restricted vector provided by Dr. B. Kleinstiver (MGH) with amplicons generated with 5′-CATTAAAAAAGGTGGATCCCCCAAGAAGAAGAGGAAAGTCTCGCTCGAG GCTAGCAcGATGGCCCAGGTGCAACTGGT-3′ (SEQ ID NO: 8) and 5′-GAGAAGTTTGTTGCCCGACGCGTCTTGATGGCCTCCTTGCTCACG-3′ (SEQ ID NO: 9) on the above-generated Nb containing plasmid and 5′-TGAGCAAGGAGGCCATCAAGACGCGTCGGGCAACAAACTTCTCTCTGC-3′ (SEQ ID NO: 10) and 5′-GCCACCACCTTCTGATAGGCAGCCTGCACCTGAGGAGTGCGGCGGCCGGC CCGCGCCACCACCTTCTGATAGGCAGC-3′ (SEQ ID NO: 11) on the original vector from Dr. B. Kleinstiver (MGH). To extract copGFP from our original E-NoMi construct (addgene #83357) to generate the E-NoMi-Red plasmid, we digested with SgrDI and NheI and assembled it with a PCR product generated with 5′-GTGAAGAGTATCAGAAGTGGCTACGAGGTGATGGAATTCTGTATGGTGAG CAAGGGCGAG-3′ (SEQ ID NO: 12) and 5′-ACCGCATGTTAGCAGACTTCCTCTGCCCTCTCCACTGCCGTACTTGTACAG CTCGTCCATGCC-3′ (SEQ ID NO: 13) primers from the E-NoMi construct. To generate the E-‘No’Mi-Red construct with TEV sites surrounding 3×FLAG-tag and NanoLuc we performed a XbaI restrict on NoMi-Red and introduce a 762 bp gBlock (see below) through Gibson assembly. The Gibson assembly reaction was performed according to manufacture standard guidelines.

Production of cargo-loaded EVs. HEK293T cells either transfected or transduced with E-NoMi-Red or E-‘No’Mi-Red containing plasmids were plated at 50% confluency in a 15-cm petri dish and incubated over night, after which the anti-mCherry nanobody with Cre plasmid was transfected using Lipofectamine 2000 (15 uL) and 3,000 ng of plasmid. Three days post-transfection, the conditioned cell culture media were collected and centrifuged at 400 g for 10 minutes to remove cellular debris. Culture medium was then concentrated using Amicon Ultra 100k spin filters (6000 g, 15 min) to a final volume of 500 uL.

EVs Isolation. The concentrated culture medium was incubated with 50 μL of anti-FLAG M2 beads at 4° C. overnight on a HulaMixer. Following incubation with magnetic immunocapture beads, NoMi-Red EVs or ‘No’Mi-Red EVs that were pulled down onto the beads were separated from non-adhered to the beads using a magnetic rack, and the beads were washed 2× with PBS. EVs were released from beads using M2 FLAG Peptide diluted in PBS for 2 hours at RT on a HulaMixer.

TEV protease recovery of No Mi-Red EVs from immunocapture beads. 10 μL of Tobacco Etch Virus (TEV) Protease (New England BioLabs) was added to anti-FLAG-selected EVs to a final volume of 500 μL and incubated overnight at 4° C. on a HulaMixer. TEV protease-released EVs were then filtered with an Amicon mini 100k spin filter to extract the TEV protease from the EVs solution.

Bioluminescence Assay. To determine the incorporation of the Nanoluc reporter derived from the E-NoMi construct in EVs, bioluminescence was measured using Furimazine (Nano-Glo Luciferase substrate, Promega, Madison, WI, USA), at a dilution of 1:500 in PBS. 5 μL of EV sample was diluted in 95 μL of PBS and added to a 96-well white Lumitrac plate. 50 μL of diluted substrate was then added to each well, and bioluminescence was measured using the Synergy H1 Hybrid Multi-Mode Reader (BioTek, Oak Ridge North, TX, USA).

EV assessment with NTA: We performed a similar method as previously published (Maalouf et al., 2023). In short, EV samples were diluted with 1×PBS to a final volume of 500 μL. Settings were adjusted according to the manufacturer's software manual (NanoSight LM10 and NTA 3.2, Malvern, UK): camera level was increased until all particles were distinctly visible. The detection threshold was set between 5 and 10. Autofocus was adjusted so that indistinct particles were avoided. For each measurement, five 1-min videos were captured, after which all the videos were analyzed by the in-built NanoSight Software NTA 3.2.

ExoView assessment of EVs. Single-EV analysis using Exoview was performed according to the guidelines provided by Unchained Labs.

Gene expression measurement. cDNAs for gene expression analysis with RT-qPCR were prepared using the SuperScript VILO cDNA Synthesis Kit (Invitrogen). qPCR mix was prepared following manufacturing protocol of Power SYBR Green PCR Master Mix (Applied Biosystems). qPCR was performed using the QuantStudio 3 PCR system (Applied Biosystems). The cycling conditions used were 50° C. for 2 min, 95° C. for 10 min, and 40 cycles of 95° C. for 15 s and 60° C. for 1 min following dissociation analysis. All qPCR reactions were done in triplicate and normalized to b-Actin mRNA levels.

Western Blot. 30 μL of NoMi-Red EVs were mixed with 10 μL of Laemmli SDS-Sample buffer (Boston BioProducts) and loaded on NuPage 4%-12% Bis-Tris polyacrylamide gels (ThermoFisher Scientific) in NuPage MES SDS Running Buffer (ThermoFisher Scientific). Transfer onto a nitrocellulose membrane was facilitated using the iBlot2 (ThermoFisher Scientific), and the membrane was blocked using 2.5% non-fat dry milk (Lab Scientific, Danvers MA) in Tris-buffered Saline (pH 7.4). Tris-buffered Saline with 0.05% Tween 20 (TBS-T) was used for primary antibody probing. The antibody probing solution was prepared with a dilution of 1:1000 in TBS-T supplemented with 10% non-fat milk solution, as previously done, and incubated overnight at 4° C. on a shaker. The membrane was washed 3 times, for 5 minutes each, with TBS-T, then incubated for 1 hour with secondary antibody and rewashed. Membranes were developed with Femto staining (ThermoFisher Scientific) and imaged using an Azure Biosystems C300 gel imager.

### Example 2—Constructing Anti-mCherry Nanobody Fused Payloads

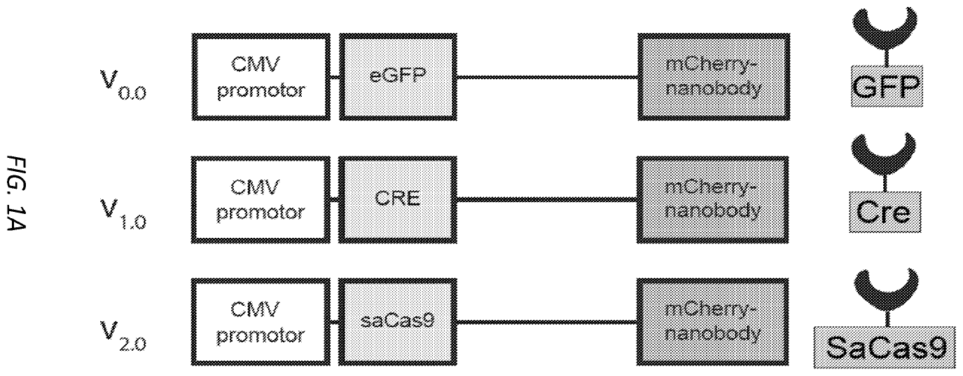

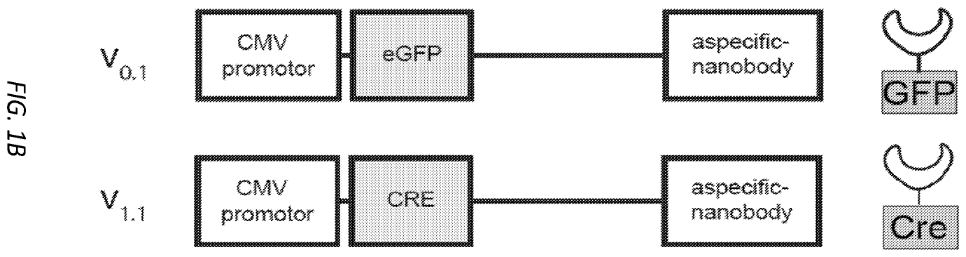

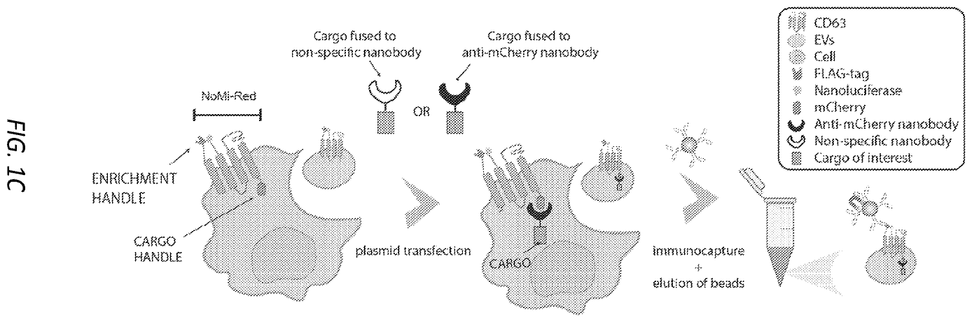

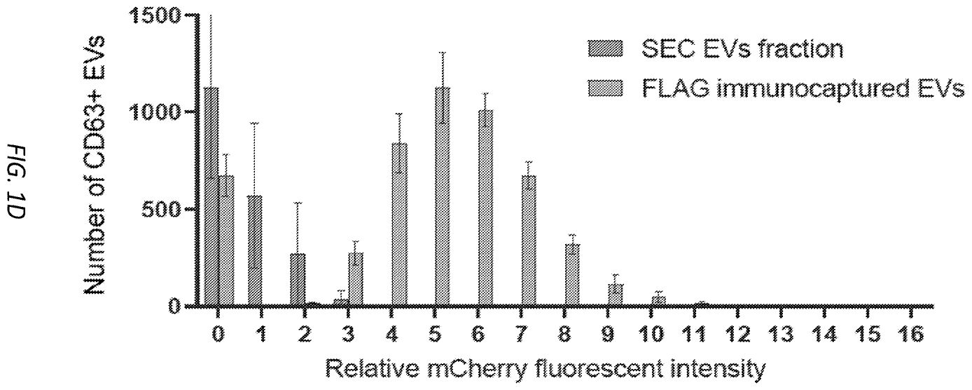

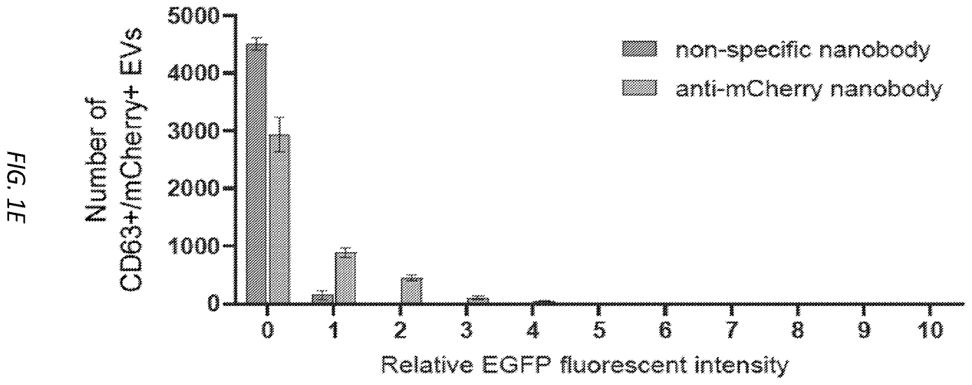

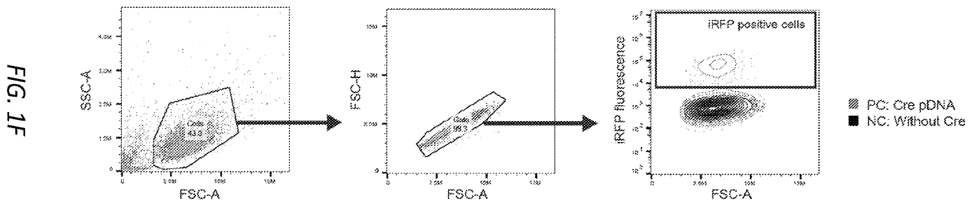

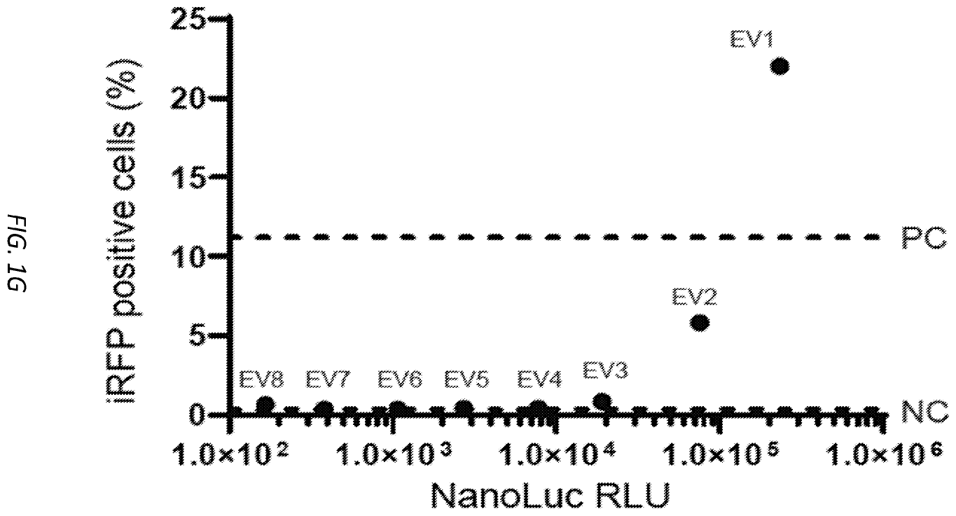

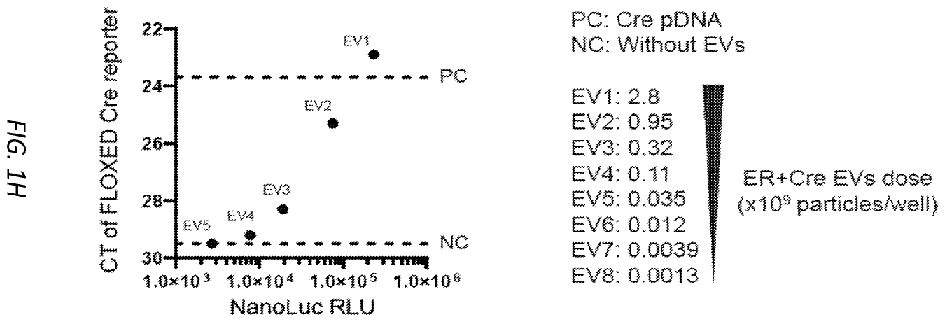

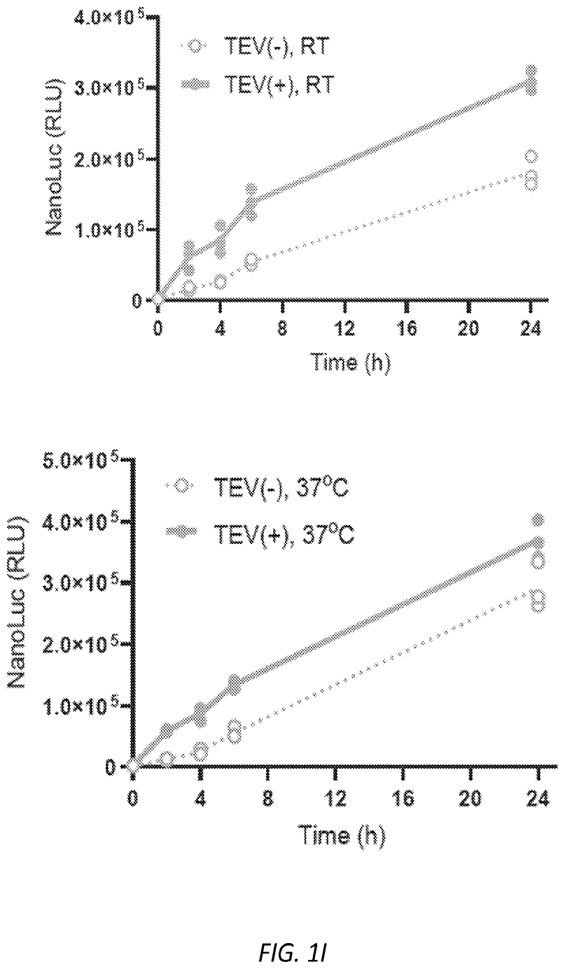

To explore whether the E-NoMi construct could be used as a tool to enrich payload-carrying EVs, vectors were constructed with transgenes encoding multiple protein cargos, including eGFP, CRE recombinase, and KKH-SaCas9, all fused to an anti-mCherry nanobody (Nb) (FIG. 1A). As controls, transgene variants were generated with eGFP and CRE fused to a Nb that does not target mCherry (FIG. 1B). To avoid any potential confusion arising from the copGFP fluorescence incorporated in the Nomi, these constructs were employed in combination with an updated version of the initial E-NoMi construct (pCDH-EF1-E-NoMi-P2A-CopGFP-T2A-PuroR). In this new version, referred to as “E-NoMi-Red”, the P2A-copGFP element was excluded, ensuring that copGFP fluorescence would not interfere with the assessment of GFP loading in the Nb-fused cargo. The strategy for isolating NoMi-Red EVs loaded with cargo involves introducing both anti-mCherry Nb-fused cargo and E-NoMi-Red expression plasmids into cells (FIG. 1C). Subsequently, these EVs were isolated using a one-step procedure, employing bead-based FLAG-tag immunocapture, which effectively eliminates contaminants such as free proteins and EVs without cargo loading.

In the following text, the term “E-NoMi-Red” refers to the genetic construct, where “E” denotes the EF-1alpha promoter, and the term “NoMi-Red” will be used for the recombinant protein found in the EVs.

### Example 3—NoMi-Red Provides a Luminal Tether for Anti-mCherry Nanobody Fused Payloads

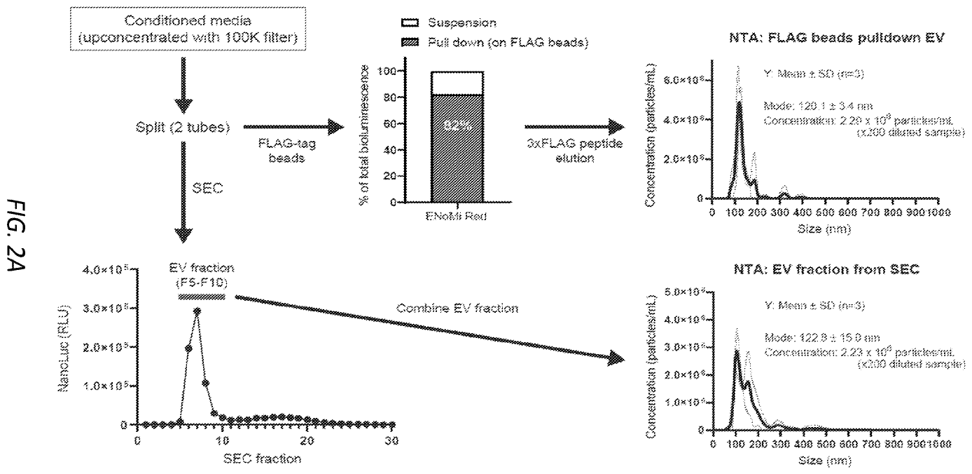

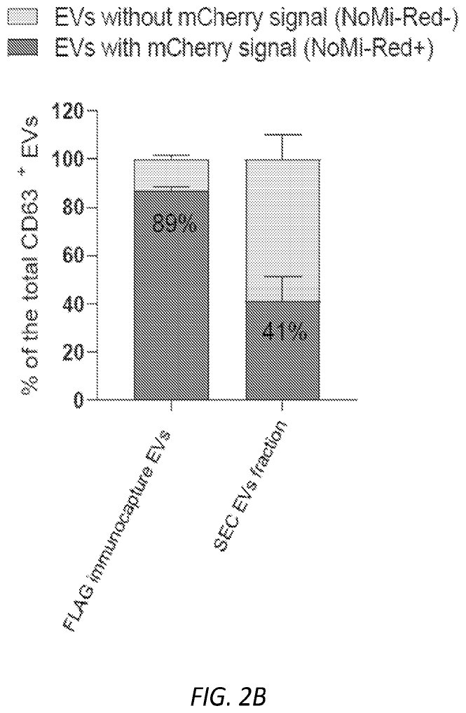

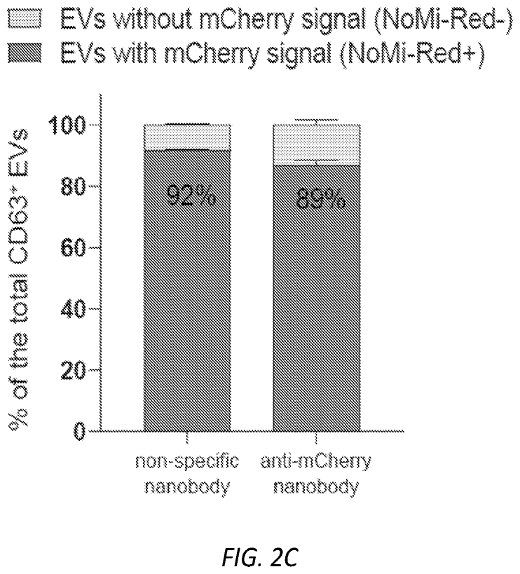

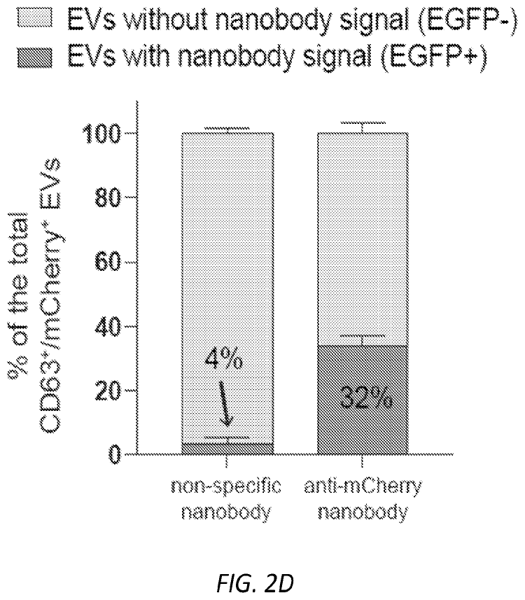

Media of E-NoMi-Red expressing cells was isolated through either size-exclusion chromatography (SEC) or anti-FLAG tag immunocapture (FIG. 2A). Nanoluciferase (Nanoluc) is displayed on the surface of NoMi-Red EVs next to the 3×FLAG tag and is used as a sensitive readout for isolating NoMi-Red-expressing EVs. In the SEC sample, the presence of Nanoluc in SEC fractions 5 to 10 was observed, which correspond to EVs. In contrast, its presence was less pronounced in the subsequent SEC fractions, which primarily contain smaller molecules from the cell media, including proteins. By incorporating the 3×FLAG tag on the surface of NoMi-Red-EVs, ˜80% of the EVs of interest were captured. This percentage was generated by comparing the Nanoluc signal retained on anti-FLAG tag affinity beads compared to the non-retained Nanoluc signal in suspension. The signal was recovered using 3×Flag-elution peptides from the beads. Nanotracking (NTA) particle analysis further confirmed that the EVs bound to the beads fell within the expected size range for EVs (˜120 nm).

Next, whether immunocapture was able to enrich NoMi-Red EVs to a greater extent than the standard non-selective SEC method was assessed. Using the Exoview™ platform, NoMi-Red EVs with mCherry fluorescence in EV solutions obtained through SEC or FLAG-tag immunocapture were quantified (FIG. 2B). EVs bound to the Exoview™ chip were visualized using anti-CD63 antibodies. Both conditions had comparable quantities of CD63-positive EVs, ensuring sample comparability. However, the SEC-isolated sample contained a greater number of EVs lacking mCherry fluorescence (59% mCherry negative EVs) compared to the expressing EVs obtained through the immunocapture method (14% mCherry negative EVs). Additionally, in the immunocapture sample, an increase in relative fluorescence intensity (RFI) per EV was detected, indicating a higher number of mCherry molecules per EV (FIG. 1D).

Next, whether immunocapture could effectively enrich EVs carrying anti-mCherry Nb-fused cargo was investigated. NoMi-Red EVs were isolated via immunocapture from E-NoMi-Red cells expressing either eGFP fused to a non-specific Nb or an anti-mCherry Nb (FIGS. 2C and 2D). Both samples showed a similar abundance of NoMi-Red EVs (comprising 80-90% of all CD63-positive EVs) bound to an Exoview™ chip, suggesting that the expression of Nb-tethered cargo did not affect NoMi-Red EV secretion and isolation. To gauge the level of cargo loading into Nb-tethered cargo-carrying EVs, the number of CD63/mCherry/GFP-triple-positive EVs was quantified. This analysis revealed a 28% higher cargo loading with the anti-mCherry Nb compared to the non-specific Nb, highlighting that Nb affinity led to increased cargo loading into NoMi-Red EVs. Additionally, the RFI per EV was analyzed to estimate the number of GFP molecules per EV (FIG. 1E). Three times higher RFI in GFP with the anti-mCherry Nb were observed compared to the non-specific Nb.

The data illustrate that an enrichment handle (i.e., 3×FLAG-tag) displayed on the surface of the EV can aid in selecting Nb-fused cargo in the EV lumen that is not covalently bound to the membrane of the EV.

### Example 4—Quantitative Assessment of NoMi-Red EVs Necessary for Functional Cargo Transfer into Recipient Cells

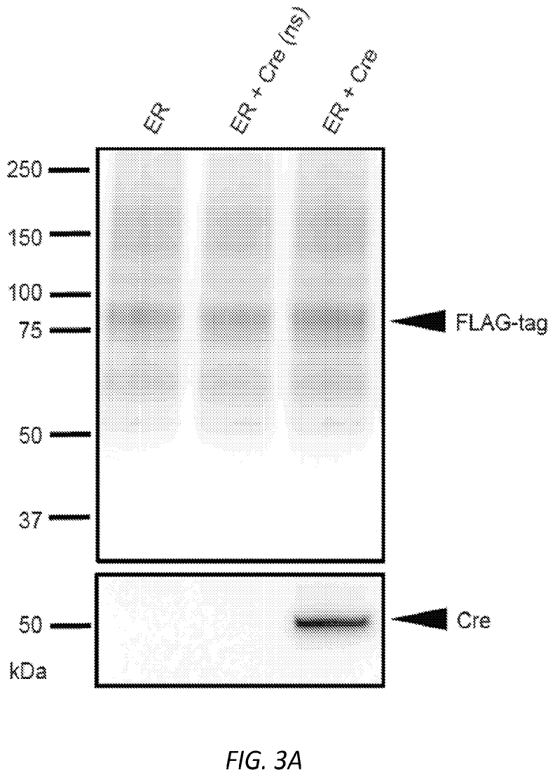

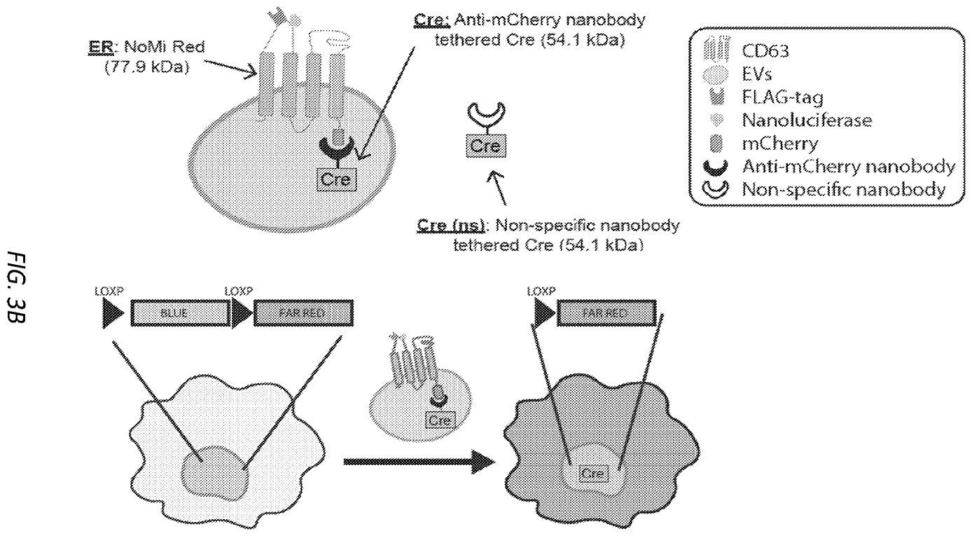

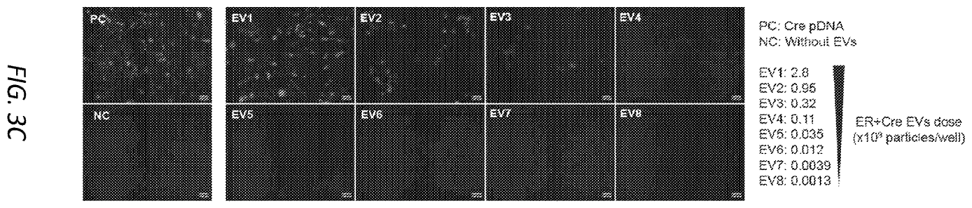

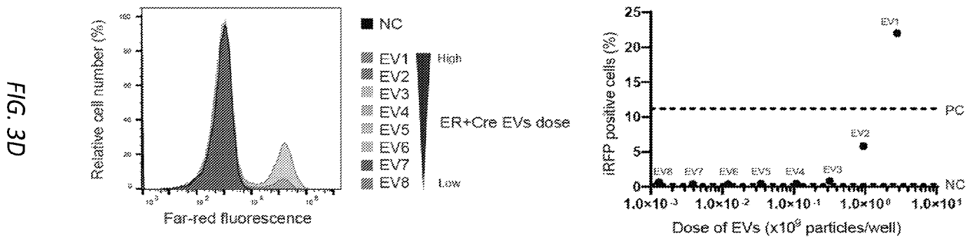

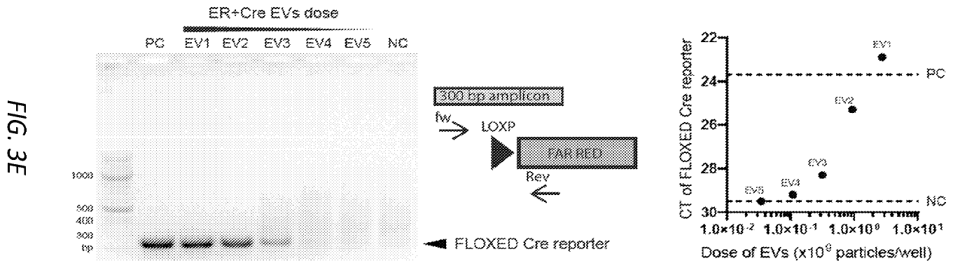

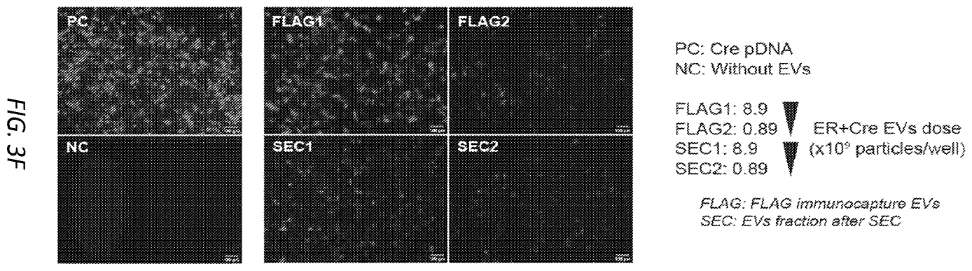

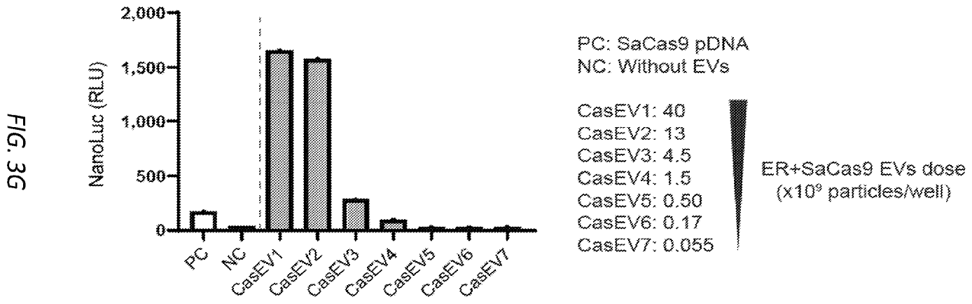

NoMi-Red EVs were isolated with immunocapture from media derived from E-NoMi-Red-expressing cells, both with and without cargo (FIG. 3A). The latter consisted of Cre tethered to a non-specific Nb or a specific Nb. Through western blotting, we observed that extracted NoMi-Red EVs contained Cre only when it was tethered to a specific Nb. This implies that mere overexpression of Cre is not sufficient for isolating E-Cre-loaded EVs compared to the immunocapture method with targeted loading. It also suggests the transferring recombinant protein rather than Cre-encoded RNA or DNA, as non-specific Nb-expressing cells should load these latter biomolecules into EVs to a similar extent as the anti-mCherry Nb.

To determine the minimum quantity of Cre-loaded E-NoMi-Red EVs required for a functional effect on recipient cells, EVs were transfected into reporter cells that undergo a color change from blue to far-red fluorescence upon successful Cre delivery (FIG. 3B). The quantity of Cre-loaded E-NoMi-Red EVs isolated through immunocapture using both NTA was assessed, providing an accurate estimate of the vesicle count added to cells, and Nanoluc, which is an indicator of the E-NoMi protein quantity in the sample. Cre-reporter cells transfected with increasing doses of Cre-loaded E-NoMi-Red EVs (ranging from 2.8×10\hat{ }9 to 1.3×10\hat{ }6 EVs as estimated by NTA) were evaluated through immunohistochemistry (IHC) (FIG. 3C), flow cytometry (FIGS. 3D, 1F, 1G), and RT-qPCR (FIGS. 3E, 1G). IHC enabled the identification of far-red positive cells when exposed to a minimum dose of 3.2×10\hat{ }8 Cre-loaded E-NoMi-Red EVs. Flow cytometry revealed that approximately 22% to 6% of the cells were affected when exposed to 2.8×10\hat{ }9 to 9.5×10\hat{ }8 EVs, respectively.

To confirm that the appearance of far-red fluorescence resulted from Cre reactivity on the LoxP sites flanking the BFP transgene, RT-qPCR was conducted. The detection of a floxed-specific amplicon occurred when a minimum of 1.1×10\hat{ }8 EVs were introduced into reporter cells, as compared to a negative control consisting of reporter cells without EV exposure. Notably, RT-qPCR was the only method that detected an effect of 1.1×10\hat{ }8 Cre-loaded E-NoMi-Red EVs, likely due to its higher sensitivity compared to IHC and flow cytometry.

We investigated the potential advantages of enriching Cre-loaded EVs through immunocapture compared to the standard isolation of EVs. Cell media collected from E-NoMi-Red expressing cells was divided to isolate EVs using either immunocapture or SEC (FIG. 3F). Subsequently, two different doses, 8.9×10\hat{ }9 EVs and 8.9×10\hat{ }8 EVs, were transfected into reporter cells. Interestingly, both doses resulted in a significantly higher number of cells exhibiting far-red fluorescence when using EVs obtained through immunocapture isolation compared to SEC isolation. This suggests that the enrichment of cargo-loaded EVs through selective immunocapture offers an additional advantage over non-selective EV isolation methods.

The technique is also feasible with cargo other than eGFP or CRE (FIG. 3G). EVs loaded with KKH-SaCas9 through a mCherry-specific Nb fusion protein were transfected into reporter cells. The reporter was activated with a minimum of 1.5×10\hat{ }9 EVs. This indicates that EVs are able to transfer multiple biomolecules to recipient cells after enrichment with E-NoMi-Red. This suggests immunocapture of E-NoMi-Red EVs are able to select EVs that have pre-made recombinant payloads that are functional in recipient cells.

### Example 5—Preserving EV's Potential as a Low-Immunogenic Non-Viral Vector by Removal of the Antigenic Loop of NoMi-Red EVs

EVs are often represented as having low or negligible immunogenicity, particularly when they originate from the same cells as the host that will receive them upon injection. Cargo-loaded E-NoMi-Red EVs immunocapture isolation uses 3×FLAG peptide as a unique tag on the EV surface to separate them from non-E-NoMi-Red EVs or free proteins. This presents a potential issue for utilizing cargo-loaded E-NoMi-Red EVs in therapy, as the 3×FLAG tag can serve as an antigen, potentially triggering an undesired immune response that may lead to increased clearance, reduced target delivery, local inflammation, or other adverse effects in patients. To develop a “scarless” method, two tobacco etch virus (TEV) protease cleavage sites flanking the 3×FLAG tag and Nanoluc-encoded sequence of E-NoMi-Red were genetically introduced (FIG. 4A). These TEV protease cleavage sites are sensitive to TEV protease activity, allowing for the release of a 24 kDa loop from the surface of the NoMi-Red EVs (FIG. 4B). In this modified setup, the 3×FLAG tag can still be utilized to select cargo-loaded NoMi-Red EVs, but the release of cargo-loaded EVs after immunocapture can be achieved by adding TEV protease instead of a FLAG tag elution peptide used in previous experiments (FIG. 4C). The 24 kDa loop is retained by the immunocapture beads to prevent the presence of antigenic 3×FLAG tag in our final EV sample. Effective removal of the 3×FLAG tag can be assessed through Nanoluc luminescence, as both components will remain attached to the immunocapture beads after TEV protease action.

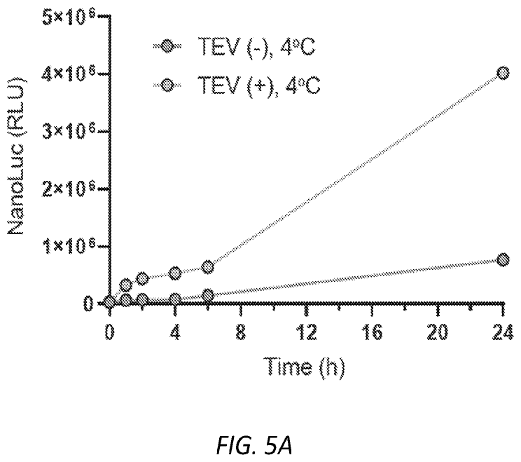

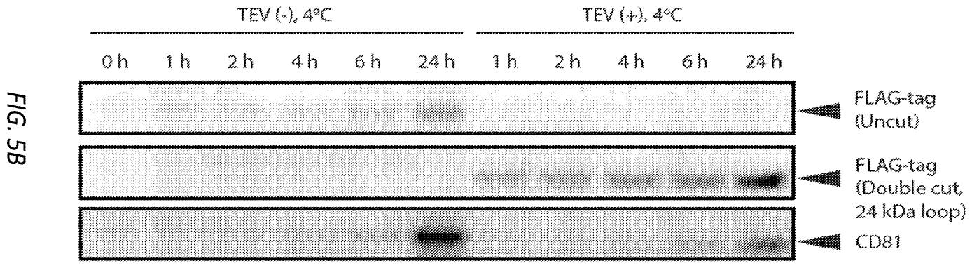

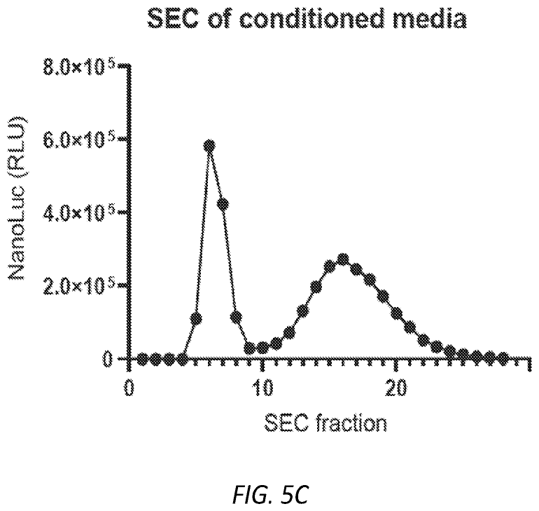

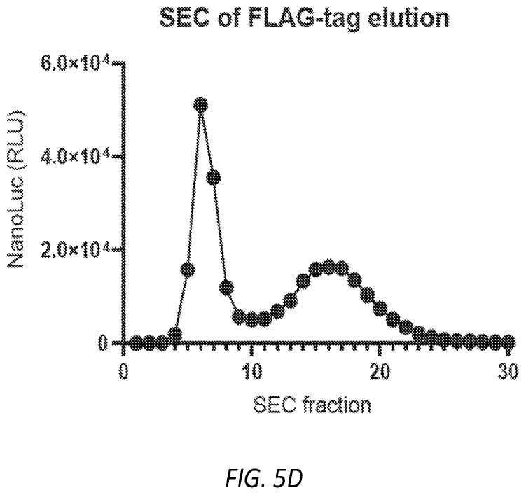

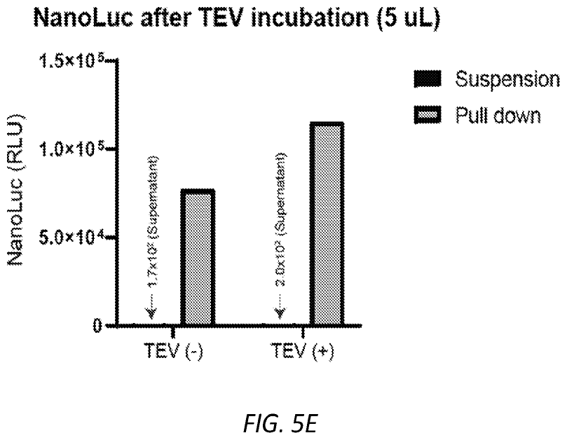

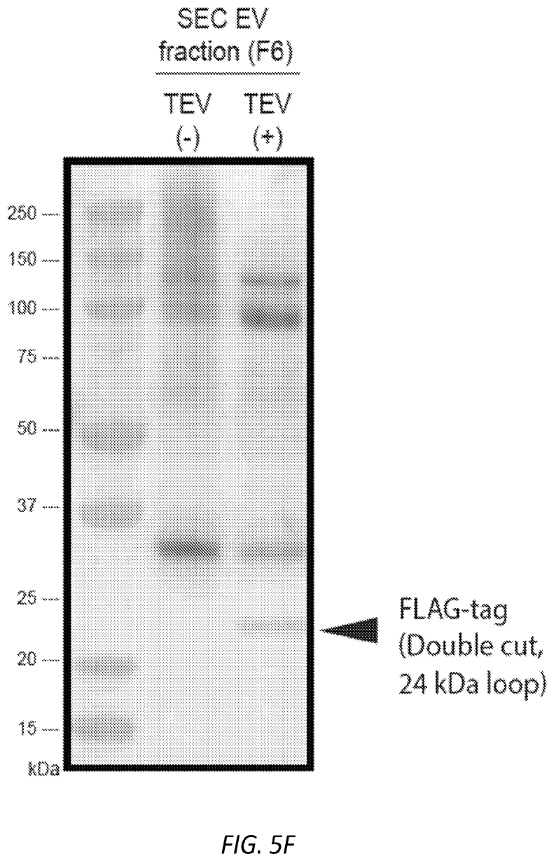

TEV protease activity at various temperatures (4° C., room temperature, and 37° C.) was evaluated to determine the optimal conditions for the new E-‘No’Mi-Red construct, with ′ representing the position of the TEV cleavage sites (FIGS. 5A, 5B, 1H). As a high-throughput screening approach, E-‘No’Mi-Red-expressing cell suspension cultures were exposed to TEV protease, and media was extracted at multiple time points (0 h, 1 h, 2 h, 4 h, 6 h, 24 h) to assess the detachment of the 24 kDa loop from the cell surface using Nanoluc (FIG. 5A) and western blot (FIG. 5B) for detection. After just 1 hour in all conditions, higher bioluminescence levels with TEV (TEV+ in FIG. 5A) was observed compared to the samples without TEV (TEV− in FIG. 5A). Western blot analysis with an anti-FLAG antibody confirmed that this increase in Nanoluc signal in the cell secretome was attributed to the detachment of the 24 kDa loop in the TEV+ sample (FIG. 5B). In the TEV− sample, the 24 kDa loop was not detected, but a gradual increase in anti-FLAG antibody signal at 78 kDa was observed, which is the predicted size of the uncleaved E-‘No’Mi-Red protein. Of note, our data indicated the time-dependent secretion of E-‘No’Mi-Red EVs by the E-‘No’Mi-Red-expressing cells, which was validated by the western blot using an anti-CD81 antibody, exhibiting a similar pattern to that of the 78 kDa uncleaved ‘No’Mi-Red protein.

Whether ‘No’Mi-Red-positive EVs are present and can be isolated from our E-‘No’Mi-Red-expressing cell secretome was explored. The majority of the Nanoluc signal was observed in the initial SEC fractions representing EVs, while the later SEC fractions, representing smaller non-EV particulates, displayed minimal signal (FIG. 5C). Further, when anti-FLAG-tag beads were exposed to E-‘No’Mi-Red-expressing cell secretions, they effectively captured the smaller particulates bearing Nanoluc signal, as evidenced by the bioluminescent signal profile observed following FLAG-tag elution peptide treatment and SEC separation (FIG. 5D). However, when immunocaptured ‘No’Mi-Red EVs were treated with TEV protease, limited Nanoluc signal was detected in the suspension, indicating that the TEV protease treated EVs had been depleted of the antigenic 24 kDa loop (FIG. 5E). This was further confirmed by western blot analysis of ‘No’Mi-Red EVs, which revealed that loop detachment, required for EV recovery after immunocapture with TEV protease, resulted in the absence of the 24 kDa band (FIG. 5F).

Thus, it is possible to enrich cargo-containing EVs with immunocapturing without exposing potential antigenic peptides on the surface of EVs in the final isolation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A fusion protein construct comprising:
an antigenic peptide sequence linked to one or more transmembrane domains, a protease cleavage motif disposed between the antigenic peptide and the one or more transmembrane domains, and a fluorescent protein, wherein when the fusion protein is expressed in a cell or inserted into the membrane of an extracellular vesicle (EV), the antigenic peptide is displayed on the outside of the plasma membrane and the fluorescent protein is present in the internal space of the EV.

2. The fusion protein construct of claim 1, wherein the antigenic peptide sequence is selected from the group consisting of an ALFA-tag sequence, an AviTag sequence, a C-tag sequence, a Calmodulin-tag sequence, an intein Capture Tag sequence, a polyglutamate tag sequence, a polyarginine tag sequence, an E-tag sequence, a FLAG-tag sequence, a 3×FLAG-tag sequence, an HA-tag sequence, a His-tag sequence, a Myc-tag sequence, a NE-tag sequence, a Rho1D4-tag sequence, a S-tag sequence, a SBP-tag sequence, a Softag1 sequence, a Softag3 sequence, a Spot-tag sequence, a Strep-tag sequence, a T7-tag sequence, a TC tag sequence, a Ty tag sequence, a V5 tag sequence, a VSV-tag sequence, a Xpress tag sequence, an isopeptag sequence, a SpyTag sequence, a SnoopTag sequence, a DogTag sequence, a SdyTag sequence, a BCCP sequence, a glutathione-S-transferase-tag sequence, a green-fluorescent protein tag sequence, a HaloTag sequence, a SNAP-tag sequence, a CLIP-tag sequence, a HUH-tag sequence, a maltose binding protein-tag sequence, a Nus-tag sequence, a Thioredoxin-tag sequence, and a Fc-tag sequence.

3. The fusion protein construct of claim 1, wherein the protease cleavage motif is selected from the group consisting of a Tobacco Etch Virus (TEV) motif, a PreScission motif, a Thrombin motif, an Xa motif, an Enterokinase motif, a Carboxypeptidase A motif, a Carboxypeptidase B motif, a Carboxypeptidase P motif, a Carboxypeptidase Y motif, a Trypsin motif, a Pepsin motif, an Elastase motif, a Papain motif, a Proteinase K motif, a Subtilisin motif, a Chymotrypsin A4 motif, a Thermolysin motif, a DAPase/Cathepsin C motif, an Endoproteinase Arg-C motif, an Endoproteinase Glu-C motif, an Endoproteinase Lys-C motif, an Endoproteinase Asp-N motif, an Acylamino-acid releasing enzyme motif, a Pyroglutamate Aminopeptidase motif, and/or a matrix metalloproteinase motif.

4. The fusion protein construct of claim 1, wherein the fluorescent protein is selected from the group consisting of mCherry, a green fluorescent protein (GFP), a yellow fluorescent protein (YFP), a blue fluorescent protein (BFP), a cyan fluorescent protein (CFP), an orange fluorescent protein (OFP), and a red fluorescent protein (RFP), or a luminescent protein.

5. The fusion protein construct of claim 4, wherein the fluorescent protein comprises mCherry.

6. The fusion protein construct of claim 1, wherein the transmembrane domain comprises tetraspanin CD63.

7. A method of generating a population of extracellular vesicles enriched for a hypoimmunogenic fusion protein construct comprising at least one transmembrane domain, and a fluorescent protein and a cargo of interest, the method comprising:
expressing the fusion protein construct of claim 1,
optionally filtering or concentrating the cell media,
incubating the cell media with antibody-coated surface, wherein the antibody of the antibody-coated surface is capable of binding the antigenic peptide of the fusion protein construct,
after incubating the cell media with antibody-coated surface, incubating the antibody-coated surface with a protease, wherein the protease cleaves the antigenic peptide from the fusion protein construct,
thereby generating the population of extracellular vesicles enriched for the hypoimmunogenic fusion protein construct comprising at least one transmembrane domain, and a fluorescent protein, and a cargo of interest.

8. The method of claim 7, further comprising introducing one or more vectors to a cell in cell media, wherein the one or more vectors express a fusion protein construct and a cargo of interest, wherein the fusion protein construct comprises an antigenic peptide sequence linked to one or more transmembrane domains, a protease cleavage motif disposed between the antigenic peptide sequence and at least one of the one or more transmembrane domains, and a fluorescent protein.

9. The method of claim 7, wherein the fluorescent protein comprises mCherry.

10. The method of claim 7, wherein the antigenic peptide sequence comprises a FLAG tag, optionally a 3×FLAG tag.

11. The method of claim 7, wherein the protease cleavage motif comprises a Tobacco Etch Virus (TEV) motif.

12. The method of claim 7, wherein the antibody-coated beads comprise an anti-FLAG antibody.

13. The method of claim 7, wherein the protease comprises Tobacco Etch Virus (TEV).

14. The method of claim 7, wherein filtering the cell media comprises using size-exclusion chromatography (SEC).

15. The method of claim 7, wherein the cargo is linked to an antibody or antibody fragment.

16. The method of claim 7, wherein the cargo is linked to a nanobody.

17. A method of delivering a cargo to a cell comprising:
administering to a subject in need thereof a therapeutically effective amount of a population of extracellular vesicles enriched for a hypoimmunogenic fusion protein construct using the method of claim 7.

18. The method of claim 17, wherein the administering is intravenous administration.

19. A cell comprising the fusion protein of claim 1, optionally further comprising an antibody linked to a cargo of interest.

20. The cell of claim 19, wherein the antibody is a nanobody.

21. The cell of claim 19, wherein the cargo of interest is an enzyme selected from the group consisting of a site-specific recombinase, a transposase, a prime editor, a base editor, a Fanzor, a transcription activator-like effector nuclease (TALEN), or a zinc-finger nuclease (ZFN).

22. The cell of claim 19, wherein the enzyme comprises Cre recombinase.

23. The cell of claim 19, wherein the cargo of interest is a CRISPR/Cas component.

24. The cell of claim 19, wherein the CRISPR/Cas component is selected from SpCas9, SaCas9, StCas9, NmCas9, FnCas9, CjCas9, ScCas9, SpCas9-HF1, eSpCas9, HypaCas9, Fok1-Fused dCas9, xCas9, SpRY/SpG, dCas9Cas4, Cas1, Cas2, Cas3, CasX, CasY, Cas12a/Cpf1, Cas12b, Cas14a, Cas13a, Cas13b, Cas13d, and base editors, wherein the CRISPR/Cas component can be complexed with an appropriate guide RNA.


SEQUENCE LISTING (14 sequences)

SEQ ID NO: 1 - DNA, 514 bp, synthetic construct
  source 1..514: mol_type = other DNA
ttcgtgaccgccgccgggatcactctcggcatggacgagctgtacaaggagaacctctactttcagtccggcgcgcctatggcccaggtgcagctggtggagagcggcggcggcctggtgcaggccggcggcagcctgagactgagctgcgccaccagcggcttcaccttcagcgactacgccatgggctggttcagacaggcccccggcaaggagagagagttcgtggccgccatcagctggagcggccacgtgaccgactacgccgacagcgtgaagggcagattcaccatcagcagagacaacgtgaagaacaccgtgtacctgcagatgaacagcctgaagcccgaggacaccgccgtgtacagctgcgccgccgccaagagcggcacctggtggtaccagagaagcgagaacgacttcggcagctggggccagggcacccaggtgaccgtgagcaaggaggccatctgatcgacaatcaacctctggattacaaaatttgtgaaagatt

SEQ ID NO: 2 - DNA, 481 bp, synthetic construct
  source 1..481: mol_type = other DNA
ttcgtgaccgccgccgggatcactctcggcatggacgagctgtacaaggagaacctctactttcagtccggcgcgcctgtgcagctgcaggagagcggcggcggcctggtgcagcccggcggcagcctgagactgagctgcaccgccagcggcgtgaccatcagcgccctgaacgccatggccatgggctggtacagacaggcccccggcgagagaagagtgatggtggccgccgtgagcgagagaggcaacgccatgtacagagagagcgtgcagggcagattcaccgtgaccagagacttcaccaacaagatggtgagcctgcagatggacaacctgaagcccgaggacaccgccgtgtactactgccacgtgctggaggacagagtggacagcttccacgactactggggccagggcacccaggtgaccgtgagcagctcgacaatcaacctctggattacaaaatttgtgaaagatt

SEQ ID NO: 3 - DNA, 762 bp, synthetic construct
  source 1..762: mol_type = other DNA
gtgggtgtcggggcacagcttgtcctgagtcagaccatatctagaattcctagggagaacctctactttcagtccgactacaaagaccatgacggtgattataaagatcatgacatcgattacaaggatgacgatgacaagggtggttctggtggtggttctggtcgatccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggagaacctctactttcagtccggcgcgccttctagaatccagggggctacccctggctctctgttgccagtggtc

SEQ ID NO: 4 - DNA, 63 bp, synthetic construct
  source 1..63: mol_type = other DNA
cacgctgttttgacctccatagaagacaccgactctagagatgcccaagaagaagaggaaggt

SEQ ID NO: 5 - DNA, 68 bp, synthetic construct
  source 1..68: mol_type = other DNA
ggccgccgccagattccaccagttgcacctgggccatgggggggtgtatatcgccatcttccagcagg

SEQ ID NO: 6 - DNA, 62 bp, synthetic construct
  source 1..62: mol_type = other DNA
cacgctgttttgacctccatagaagacaccgactctagaatgcccaagaagaagaggaaggt

SEQ ID NO: 7 - DNA, 59 bp, synthetic construct
  source 1..59: mol_type = other DNA
gctgcaccaggccgccgccgctctcctgcagctgcacaggatcgccatcttccagcagg

SEQ ID NO: 8 - DNA, 78 bp, synthetic construct
  source 1..78: mol_type = other DNA
cattaaaaaaggtggatcccccaagaagaagaggaaagtctcgctcgaggctagcacgatggcccaggtgcaactggt

SEQ ID NO: 9 - DNA, 45 bp, synthetic construct
  source 1..45: mol_type = other DNA
gagaagtttgttgcccgacgcgtcttgatggcctccttgctcacg

SEQ ID NO: 10 - DNA, 48 bp, synthetic construct
  source 1..48: mol_type = other DNA
tgagcaaggaggccatcaagacgcgtcgggcaacaaacttctctctgc

SEQ ID NO: 11 - DNA, 77 bp, synthetic construct
  source 1..77: mol_type = other DNA
gccaccaccttctgataggcagcctgcacctgaggagtgcggcggccggcccgcgccaccaccttctgataggcagc

SEQ ID NO: 12 - DNA, 60 bp, synthetic construct
  source 1..60: mol_type = other DNA
gtgaagagtatcagaagtggctacgaggtgatggaattctgtatggtgagcaagggcgag

SEQ ID NO: 13 - DNA, 63 bp, synthetic construct
  source 1..63: mol_type = other DNA
accgcatgttagcagacttcctctgccctctccactgccgtacttgtacagctcgtccatgcc

SEQ ID NO: 14 - protein, 7 aa, unidentified
ENLYFQS